SEQUENCE LISTING (22 sequences)

SEQ ID NO: 1 - protein, 452 aa, Artificial Sequence
  Bermekimab heavy chain
QVQLVESGGGVVQPGRSLRLSCTASGFTFSMFGVHWVRQAPGKGLEWVAAVSYDGSNKYYAESVKGRFTISRDNSKNILFLQMDSLRLEDTAVYYCARGRPKVVIPAPLAHWGQGTLVTFSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, Artificial Sequence
  Bermekimab light chain
DIQMTQSPSSVSASVGDRVTITCRASQGISSWLAWYQQKPGKAPKLLIYEASNLETGVPSRFSGSGSGSDFTLTISSLQPEDFATYYCQQTSSFLLSFGGGTKVEHKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 122 aa, Artificial Sequence
  Bermekimab heavy chain variable domain
QVQLVESGGGVVQPGRSLRLSCTASGFTFSMFGVHWVRQAPGKGLEWVAAVSYDGSNKYYAESVKGRFTISRDNSKNILFLQMDSLRLEDTAVYYCARGRPKVVIPAPLAHWGQGTLVTFSS

SEQ ID NO: 4 - protein, 108 aa, Artificial Sequence
  Bermekimab light chain variable domain
DIQMTQSPSSVSASVGDRVTITCRASQGISSWLAWYQQKPGKAPKLLIYEASNLETGVPSRFSGSGSGSDFTLTISSLQPEDFATYYCQQTSSFLLSFGGGTKVEHKR

SEQ ID NO: 5 - protein, 8 aa, Artificial Sequence
  Bermekimab HCDR1 (IMGT)
GFTFSMFG

SEQ ID NO: 6 - protein, 8 aa, Artificial Sequence
  Bermekimab HCDR2 (IMGT)
VSYDGSNK

SEQ ID NO: 7 - protein, 15 aa, Artificial Sequence
  Bermekimab HCDR3 (IMGT)
ARGRPKVVIPAPLAH

SEQ ID NO: 8 - protein, 6 aa, Artificial Sequence
  Bermekimab LCDR1 (IMGT)
QGISSW

SEQ ID NO: 9 - protein, 3 aa, Artificial Sequence
  Bermekimab LCDR2 (IMGT)
EAS

SEQ ID NO: 10 - protein, 9 aa, Artificial Sequence
  Bermekimab LCDR3 (IMGT)
QQTSSFLLS

SEQ ID NO: 11 - protein, 5 aa, Artificial Sequence
  Bermekimab HCDR1 (Kabat)
MFGVH

SEQ ID NO: 12 - protein, 17 aa, Artificial Sequence
  Bermekimab HCDR2 (Kabat)
AVSYDGSNKYYAESVKG

SEQ ID NO: 13 - protein, 13 aa, Artificial Sequence
  Bermekimab HCDR3 (Kabat)
GRPKVVIPAPLAH

SEQ ID NO: 14 - protein, 11 aa, Artificial Sequence
  Bermekimab LCDR1 (Kabat)
RASQGISSWLA

SEQ ID NO: 15 - protein, 7 aa, Artificial Sequence
  Bermekimab LCDR2 (Kabat)
EASNLET

SEQ ID NO: 16 - protein, 9 aa, Artificial Sequence
  Bermekimab LCDR3 (Kabat)
QQTSSFLLS

SEQ ID NO: 17 - protein, 7 aa, Artificial Sequence
  Bermekimab HCDR1 (Chothia)
GFTFSMF

SEQ ID NO: 18 - protein, 6 aa, Artificial Sequence
  Bermekimab HCDR2 (Chothia)
SYDGSN

SEQ ID NO: 19 - protein, 13 aa, Artificial Sequence
  Bermekimab HCDR3 (Chothia)
GRPKVVIPAPLAH

SEQ ID NO: 20 - protein, 11 aa, Artificial Sequence
  Bermekimab LCDR1 (Chothia)
RASQGISSWLA

SEQ ID NO: 21 - protein, 7 aa, Artificial Sequence
  Bermekimab LCDR2 (Chothia)
EASNLET

SEQ ID NO: 22 - protein, 9 aa, Artificial Sequence
  Bermekimab LCDR3 (Chothia)
QQTSSFLLS

(19) United States
(12) Patent Application Publication
(10) Pub. No.: US 2023/0235042 A1
(43) Pub. Date: Jul. 27, 2023

(54) TREATMENT OF HIDRADENITIS SUPPURATIVA

(71) Applicant: Janssen Biotech, Inc., Horsham, PA (US)

(72) Inventors: Yanli ZHUANG, Horsham, PA (US); Bhaskar SRIVASTAVA, Horsham, PA (US); Karen KEEFE, Horsham, PA (US); Swaroopa PARATKAR, Horsham, PA (US); Bruce RANDAZZO, Horsham, PA (US); Emesto MUNOZ, Horsham, PA (US); John SIMARD, Horsham, PA (US)

(21) Appl. No.: 17/996,099

(22) PCT Filed: Apr. 16, 2021
(86) PCT No.: PCT/US2021/027614

§ 371 Date: Oct. 13, 2022

Related U.S. Application Data
(60) Provisional application No. 63/010,923, filed on Apr. 16, 2020.

(51) Int. Cl. C07K 16/24; A61P 17/00; C07K 16/12

(52) U.S. Cl. CPC C07K 16/245; A61P 17/00; C07K 16/1271; A61K 2039/505

(57) ABSTRACT

Hidradenitis suppurativa can be treated by administering a pharmaceutical composition that includes a pharmaceutically acceptable carrier and a therapeutically effective amount of an agent that selectively binds IL-1α.

### CROSS-REFERENCE TO RELATED APPLICATIONS

The present application claims the priority of U.S. Provisional Pat. Application Serial No. 63/010,923 filed on April 16th, 2020.

### STATEMENT AS TO FEDERALLY SPONSORED RESEARCH

Not applicable.

### FIELD OF THE INVENTION

The invention relates generally to the fields of medicine, dermatology, and immunology. More particularly, the invention relates to the use of antibodies (Abs) which specifically bind interleukin-1α (IL-1α) to treat hidradenitis suppurativa.

### BACKGROUND

Hidradenitis suppurativa (HS) is a chronic debilitating skin disease where nodules appearing in areas rich in apocrine glands progressively swell until they rupture and release pus through the skin. Sinus tract formation and scars result. HS is typically treated with antibiotics and surgery, but frequent relapse drastically impairs the patient’s quality of life.

### SUMMARY

Disclosed herein is the discovery that an agent that specifically targets IL,-1α is useful for treating HS.

Accordingly, described herein are methods of reducing the severity of HS symptoms in a human subject. These methods can include the step of administering to the subject a pharmaceutical composition including a pharmaceutically acceptable carrier and an amount of an agent that selectively binds IL-1α effective to reduce the number and/or size of inflammatory lesions (e.g., nodule, abscesses, or draining fistulas), prevent their progression, reduce the pain caused by the lesions, or increase the time until new exacerbations. The agent can be an anti-IL-1α antibody (Ab) such as a monoclonal antibody (mAb) (e.g., of the IgG1 isotype), a mAb that includes a complementarity determining region (CDR) of MABp1, or MABp1.

Another aspect of the invention features a method of reducing the symptoms of HS in a human subject by administering to the subject a pharmaceutical composition including a pharmaceutically acceptable carrier and an amount of an anti-IL-1α Ab (or other agent that specifically and/or selectively binds IL-1α) effective to reduce the number and/or size of inflammatory lesions (e.g., nodule, abscesses, or draining fistulas) in the subject by at least about 10% (e.g., at least 8, 9, 10, 15, 17, 20, 30, 40, 50, 60, 70, 80, 90, or 100%) as measured by any standard dermatological test.

The anti-IL-1α Ab can be a mAb such as an IgG1. The anti-IL-1α Ab can be the mAb designated as MABp1 or a mAb that includes one or more (CDRs) of MABp1. The pharmaceutical composition can be administered to the subject by injection, infusion, subcutaneously, intravenously, intramuscularly, or intradermally. In the methods described herein, the dose can be at least 0.25 (e.g., at least 0.2, 0.5, 0.75., 1, 2, 3, 4, or 5) mg/kg, and preferably at between 1-20 mg/kg (e.g., 1, 2, 3, 4, 5, 6, 7, 8, 9, 10, 11, 12, 13, 14, 15, 16, 17, 18, 19, or 20 +/- 0.1, 0.2, 0.3, 0.4, 0.5, 0.6, 0.7, 0.8, or 0.9 mg/kg).

Unless otherwise defined, all technical terms used herein have the same meaning as commonly understood by one of ordinary skill in the art to which this invention belongs. Commonly understood definitions of biological terms can be found in Rieger et al., Glossary of Genetics: Classical and Molecular, 5th edition, Springer-Verlag: New York, 1991; and Lewin, Genes V, Oxford University Press: New York, 1994. Commonly understood definitions of medical terms can be found in Stedman’s Medical Dictionary, 27^{th} Edition, Lippincott, Williams & Wilkins, 2000.

As used herein, an “antibody” or “Ab” is an immunoglobulin (Ig), a solution of identical or heterogeneous Igs, or a mixture of Igs. An “Ab” can also refer to fragments and engineered versions of Igs such as Fab, Fab′, and F(ab′)₂ fragments; and scFv’s, heteroconjugate Abs, and similar artificial molecules that employ Ig-derived CDRs to impart antigen specificity. A “monoclonal antibody” or “mAb” is an Ab expressed by one clonal B cell line or a population of Ab molecules that contains only one species of an antigen binding site capable of immunoreacting with a particular epitope of a particular antigen. A “polyclonal Ab” is a mixture of heterogeneous Abs. Typically, a polyclonal Ab will include myriad different Ab molecules which bind a particular antigen with at least some of the different Abs immunoreacting with a different epitope of the antigen. As used herein, a polyclonal Ab can be a mixture of two or more mAbs.

An “antigen-binding portion” of an Ab is contained within the variable region of the Fab portion of an Ab and is the portion of the Ab that confers antigen specificity to the Ab (i.e., typically the three-dimensional pocket formed by the CDRs of the heavy and light chains of the Ab). A “Fab portion” or “Fab region” is the proteolytic fragment of a papain-digested Ig that contains the antigen-binding portion of that Ig. A “non-Fab portion” is that portion of an Ab not within the Fab portion, e.g., an “Fc portion” or “Fc region.” A “constant region” of an Ab is that portion of the Ab outside of the variable region. Generally encompassed within the constant region is the “effector portion” of an Ab, which is the portion of an Ab that is responsible for binding other immune system components that facilitate the immune response. Thus, for example, the site on an Ab that binds complement components or Fc receptors (not via its antigen-binding portion) is an effector portion of that Ab.

When referring to a protein molecule such as an Ab, “purified” means separated from components that naturally accompany such molecules. Typically, an Ab or protein is purified when it is at least about 10% (e.g., 9%, 10%, 20%, 30% 40%, 50%, 60%, 70%, 80%, 90%, 95%, 98%, 99%, 99.9%, and 100%), by weight, free from the non-Ab proteins or other naturally-occurring organic molecules with which it is naturally associated. Purity can be measured by any appropriate method, e.g., column chromatography, polyacrylamide gel electrophoresis, or HPLC analysis. A chemically-synthesized protein or other recombinant protein produced in a cell type other than the cell type in which it naturally occurs is “purified.”

By “bind”, “binds”, or “reacts with” is meant that one molecule recognizes and adheres to a particular second molecule in a sample, but does not substantially recognize or adhere to other molecules in the sample. Generally, an Ab that “specifically binds” another molecule has a K_{d} greater than about 10⁵, 10⁶, 10⁷, 10⁸, 10⁹, 10¹⁰, 10¹¹, or 10¹² liters/mole for that other molecule. An Ab that “selectively binds” a first molecule specifically binds the first molecule at a first epitope but does not specifically bind other molecules that do not have the first epitope. For example, an Ab which selectively binds IL-1alpha specifically binds an epitope on IL-1alpha but does not specifically bind IL-1beta (which does not have the epitope).

A “therapeutically effective amount” is an amount which is capable of producing a medically desirable effect in a treated animal or human (e.g., amelioration or prevention of a disease or symptom of a disease).

Although methods and materials similar or equivalent to those described herein can be used in the practice or testing of the present invention, suitable methods and materials are described below. All patents, patent applications, and publications mentioned herein are incorporated by reference in their entirety. In the case of conflict, the present specification, including definitions will control. In addition, the particular embodiments discussed below are illustrative only and not intended to be limiting.

### BRIEF DESCRIPTION OF DRAWINGS

FIG. 1 is a graph showing that 60% of patients allocated to treatment with MABp1 achieved positive HiSCR at week 12 compared to 10% of the placebo group; and that the odds ratio (OR) for positive HiSCR under MABp1 was 13.50 (95% confidence intervals: 1.19-152.51; p= 0.035).

FIG. 2 is a graph showing that the clinical efficacy of MABp1 was maintained until week 24 (i.e., 12 weeks after treatment was stopped), where no patients treated with placebo had a positive HiSCR score (0%) compared to four out of 10 patients (40%) treated with MABp1.

FIG. 3 is a graph showing the percent change of the total AN (sum of inflammatory nodules and abscesses) count in all patients over the first 24 weeks after the start of treatment with MABp1 or placebo.

FIG. 4 is a graph showing the percent change of the total AN count in patients without previous exposure to anti-TNFα over the first 24 weeks after the start of treatment with MABp1 or placebo.

FIG. 5 is a graph showing the percent change of the total AN count in patients with previous anti-TNFα treatment failure over the first 24 weeks after the start of treatment with MABp1 or placebo.

FIG. 6 is a graph showing the percent change in disease activity in patients without previous exposure to anti-TNFα over the first 24 weeks after the start of treatment with MABp1 or placebo.

FIG. 7 is a graph showing the percent change in visual analogue scale (VAS) in all patients over the first 24 weeks after the start of treatment with MABp1 or placebo.

FIG. 8 is a graph showing the median time to new exacerbations in patients without previous exposure to anti-TNFα over the first 24 weeks after the start of treatment with MABp1 or placebo.

FIG. 9 is a graph showing the change in lesion depth in all patients after 12 weeks from the start of treatment with MABp1 or placebo.

FIG. 10 is a graph showing the change in lesion depth in patients with previous anti-TNFα treatment failure after 12 weeks from the start of treatment with MABp1 or placebo.

FIG. 11 is a graph showing the number of patients having at least a 20% reduction in lesion depth in patients treated with MABp1 or placebo.

FIG. 12 is a graph showing the number of patients having at least a 20% reduction in lesion depth in patients treated with MABp1 or placebo, wherein the patient populations were (i) those without previous exposure to anti-TNFα and (ii) those with previous anti-TNFα treatment failure.

FIG. 13 is a chart showing prior medical history of subjects in the study described in Example 1 below.

FIG. 14 is a chart showing baseline disease severity subjects in the study described in Example 1 below.

FIG. 15 is a flowchart summarizing a confirmatory study described in Example 2 below where bermekimab (MABp1) was formulated for subcutaneous administration at 400 mg per week for 12 weeks.

FIG. 16 is a chart showing the baseline characteristics of study subjects (anti-TNF failures vs. anti-TNF naives) who participated in the study described in Example 2.

FIG. 17 is a chart summarizing the results of the study described in Example 2.

FIG. 18 is a Study calendar of the study described in Example 2.

FIGS. 19-36 are graphs showing various results of the study described in Example 2.

FIG. 37 is an updated version of FIG. 16, including statistical analyses.

FIG. 38 illustrates the statistically significant mean percent changes in inflammatory lesion count that patients in both groups A and B achieved relative to their baselines. Group A saw a 46% mean percent change from baseline (P < 0.0001) and group B saw a 60% mean percent change from baseline (P = 0.004).

FIG. 39 illustrates the percentage of subjects in group A (n = 24) and group B (n = 18) who achieved HiSCR by weeks 2, 6, and 12. Error bars shown in the figure are mean ± SEM. Achievement of HiSCR is defined as at least a 50% decrease of the total inflammatory lesion count from the baseline before start of treatment and the absence of new abscess or fistula formation.

FIG. 40 provides descriptive statistics for subjects receiving bermekimab: change at Week 12

FIG. 41 provides a patient disposition flow chart. The study consisted of two groups. Group A (n = 24): bermekimab administered subcutaneously at a dose of 400 mg weekly (13 doses) in patients who had previously failed anti-TNF therapy. Group B (n = 18): bermekimab administered subcutaneously at a dose of 400 mg weekly (13 doses) in patients who were anti-TNF naive. Patients were followed for 13 weeks to allow for assessment of safety and preliminary efficacy. PI, principal investigator.

FIG. 42 is a schematic summarizing the Phase 1 study of Example 4

FIG. 43 and FIG. 44 show the proportion differences (95% CI) between the bermekimab groups and the placebo group at weeks 12 and 16 respectively, for the study described in Example 4.

FIG. 45 shows the HiSCR, HiSCR75 and HiSCR90 response rates and 95% CI over time for the study described in Example 4.

FIG. 46 shows the change from baseline in HS-related skin pain in the Past 24 hours, AN count, and DLQI over time for the study described in Example 4.

FIG. 47 illustrates the exposure-response (E-R) relationship between HiSCR50 Response and Steady-state Trough Bermekimab Concentrations at Wks 12/16 in 400 mg qw Group for the study described in Example 4.

FIG. 48 is a schematic summarizing the design of the Phase 2b study of Example 6.

FIG. 49 is a graph showing the predicted skin free IL-1α concentrations following subcutaneous dosing of bermekimab in Hidradenitis Suppurativa subjects, based on the model described in Example 6.

### DETAILED DESCRIPTION

The invention encompasses compositions and methods for reducing skin inflammation in HS including ameliorating one or more symptoms of a dermatological pathology in a subject. The below described preferred embodiments illustrate adaptation of these compositions and methods. Nonetheless, from the description of these embodiments, other aspects of the invention can be made and/or practiced based on the description provided below.

### General Methodology

Methods involving conventional immunological and molecular biological techniques are described herein. Immunological methods (for example, assays for detection and localization of antigen-Ab complexes, immunoprecipitation, immunoblotting, and the like) are generally known in the art and described in methodology treatises such as Current Protocols in Immunology, Coligan et al., ed., John Wiley & Sons, New York. Techniques of molecular biology are described in detail in treatises such as Molecular Cloning: A Laboratory Manual, 2nd ed., vol. 1-3, Sambrook et al., ed., Cold Spring Harbor Laboratory Press, Cold Spring Harbor, N.Y., 2001; and Current Protocols in Molecular Biology, Ausubel et al., ed., Greene Publishing and Wiley-Interscience, New York. Ab methods are described in Handbook of Therapeutic Abs, Dubel, S., ed., Wiley-VCH, 2007. General methods of medical treatment are described in McPhee and Papadakis, Current Medical Diagnosis and Treatment 2010, 49^{th} Edition, McGraw-Hill Medical, 2010; and Fauci et al., Harrison’s Principles of Internal Medicine, 17^{th} Edition, McGraw-Hill Professional, 2008. Methods in dermatology are described in James et al., Andrews’ Diseases of the Skin: Clinical Dermatology - Expert Consult, 11^{th} Ed., Saunders, 2011; and Burns et al., Rook’s Textbook of Dermatology, 8^{th} Ed., Wiley-Blackwell, 2010.

### Treatment

The compositions and methods described herein are useful for treating HS in a mammalian subject by administering to the subject a pharmaceutical composition including an amount of an anti-IL-1α Ab effective to improve at least one characteristic of the condition in the subject (e.g., reduce the number and/or size of nodules, abscesses, or draining fistulas or prevent their progression), or to improve one or more of the scores described below in the Examples section by at least 10% (e.g., at least 10, 20, 30, 40, 50, 60, or 70%) or by at least one point (e.g., at least 1, 2, 3, 4, 5, 6, 7, 8, or 9 points). The mammalian subject might be any that suffers from HS including human beings. Human subjects might be male, female, adults, children, seniors (65 and older), and those with other diseases. Particularly preferred subjects are (i) those whose disease has progressed or failed to respond after treatment with other anti-inflammatory (e.g., TNFα inhibitors) or anti-microbial agents; (ii) those with a familial history of HS; (iii) those in which other anti-inflammatory (e.g., TNFα inhibitors) or anti-microbial agents are not suitable; and (iv) those with higher than 100, 200, 300, 400, 500, or 1000 pg/ml of IL-1α in pus taken from their lesions. Subjects who have developed a human anti-human antibody response due to prior administration of therapeutic antibodies are preferred when the anti-IL-1α Ab is a true human Ab (e.g., one that is naturally expressed in a human subject) such as MABp1.

In one embodiment, the subject has not received a prior treatment with an anti-TNFα antibody (for example, adalimumab). In one embodiment, the subject has received and failed to respond to prior treatment with an anti-TNFα antibody (for example, adalimumab).

### Antibodies and Other Agents That Target IL-1α

Any suitable type of Ab that specifically binds IL-1α and reduces a characteristic of HS in a subject might be used. For example, the anti-IL-1α Ab used might be mAb, a polyclonal Ab, a mixture of mAbs, or an Ab fragment or engineered Ab-like molecule such as an scFv. The Ka of the Ab is preferably at least 1 ×10⁹ M⁻¹ or greater (e.g., greater than 9 ×10¹⁰ M⁻¹, 8 ×10¹⁰ M⁻¹, 7 ×10¹⁰ M⁻¹, 6 ×10¹⁰ M⁻¹, 5 ×10¹⁰ M⁻¹, 4 ×10¹⁰ M⁻¹, 3 ×10¹⁰ M⁻¹, 2 ×10¹⁰ M⁻¹, or 1 ×10¹⁰ M⁻¹). In a preferred embodiment, the invention utilizes a fully human mAb that includes (i) an antigen-binding variable region that exhibits very high binding affinity (e.g., at least nano or picomolar) for human IL-1α and (ii) a constant region. The human Ab is preferably an IgG1, although it might be of a different isotype such as IgM, IgA, or IgE, or subclass such as IgG2, IgG3, or IgG4. One example of a particularly useful mAb is MABp1, an IL-1α-specific IgG1 mAb described in U.S. Pat. No. 8,034,337B2 issued on Oct. 11, 2011. Other useful mAbs are those that include at least one but preferably all the CDRs of MABp1. CDRs may be determined according to known methods such as described in Ofran et al., J. Immunol., 181:6230, 2008; and Antibody Engineering Volume 2, 2d edition, Konterman and Dubel (eds), Springer, 2010. Abs that specifically binds IL-1α and methods of their manufacture are described in more detail in, e.g., U.S. Pat. No. 9,545,411. Other useful mAbs are those that include the variable domains (VH and VL) of bermekimab.

The full heavy chain amino acid sequence of bermekimab is:

QVQLVESGGGVVQPGRSLRLSCTASGFTFSMFGVHWVRQAPGKGLEWVAA
VSYDGSNKYYAESVKGRFTISRDNSKNILFLQMDSLRLEDTAVYYCARGR
PKVVIPAPLAHWGQGTLVTFSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKRVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK (SEQ ID NO: 1).

The full light chain amino acid sequence of bermekimab is:

DIQMTQSPSSVSASVGDRVTITCRASQGISSWLAWYQQKPGKAPKLLIYE
ASNLETGVPSRFSGSGSGSDFTLTISSLQPEDFATYYCQQTSSFLLSFGG
GTKVEHKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC (SEQ ID NO: 2).

The heavy chain variable domain sequence of bermekimab (VH) is:

QVQLVESGGGVVQPGRSLRLSCTASGFTFSMFGVHWVRQAPGKGLEWVAA
VSYDGSNKYYAESVKGRFTISRDNSKNILFLQMDSLRLEDTAVYYCARGR
PKVVIPAPLAHWGQGTLVTFSS (SEQ ID NO: 3).

The light chain variable domain sequence of bermekimab (VL) is:

DIQMTQSPSSVSASVGDRVTITCRASQGISSWLAWYQQKPGKAPKLLIYE
ASNLETGVPSRFSGSGSGSDFTLTISSLQPEDFATYYCQQTSSFLLSFGG
GTKVEHKR (SEQ ID NO: 4).

The CDRs of the heavy chain of bermekimab, according to the IMGT definition, are:
HCDR1 : GFTFSMFG (SEQ ID NO: 5)
HCDR2 : VSYDGSNK (SEQ ID NO: 6)
HCDR3 : ARGRPKVVIPAPLAH (SEQ ID NO: 7)

The CDRs of the light chain of bermekimab, according to the IMGT definition, are:
LCDR1 : QGISSW (SEQ ID NO: 8)
LCDR2 : EAS (SEQ ID NO: 9)
LCDR3 : QQTSSFLLS (SEQ ID NO: 10)

The CDRs of the heavy chain of bermekimab, according to the Kabat definition, are:
HCDR1 : MFGVH (SEQ ID NO: 11)
HCDR2 : AVSYDGSNKYYAESVKG (SEQ ID NO: 12)
HCDR3 : GRPKVVIPAPLAH (SEQ ID NO: 13)

The CDRs of the light chain of bermekimab, according to the Kabat definition, are:
LCDR1 : RASQGISSWLA (SEQ ID NO: 14)
LCDR2 : EASNLET (SEQ ID NO: 15)
LCDR3 : QQTSSFLLS (SEQ ID NO: 16)

The CDRs of the heavy chain of bermekimab, according to the Chothia definition, are:
HCDR1 : GFTFSMF (SEQ ID NO: 17)
HCDR2 : SYDGSN (SEQ ID NO: 18)
HCDR3 : GRPKVVIPAPLAH (SEQ ID NO: 19)

The CDRs of the light chain of bermekimab, according to the Chothia definition, are:
LCDR1 : RASQGISSWLA (SEQ ID NO: 20)
LCDR2 : EASNLET (SEQ ID NO: 21)
LCDR3 : QQTSSFLLS (SEQ ID NO: 22)

In some embodiments, the anti-IL-1α Ab comprises the HCDR1, HCDR2 and HCDR3 of SEQ ID NOs: 5, 6 and 7 respectively, and the LCDR1, LCDR2 and LCDR3 of SEQ ID NOs: 8, 9 and 10.

In other embodiments, the anti-IL-1α Ab comprises the HCDR1, HCDR2 and HCDR3 of SEQ ID NOs: 11, 12 and 13 respectively, and the LCDR1, LCDR2 and LCDR3 of SEQ ID NOs: 14, 15 and 16 respectively.

In other embodiments, the anti-IL-1α Ab comprises the HCDR1, HCDR2 and HCDR3 of SEQ ID NOs: 17, 18 and 19 respectively and the LCDR1, LCDR2 and LCDR3 of SEQ ID NOs: 20, 21 and 22.

In one embodiment, the anti-IL-1α Ab comprises the VH of SEQ ID NO: 3 and the VL of SEQ ID NO: 4.

In one embodiment, the anti-IL-1α Ab comprises the HC of SEQ ID NO: 1 and the LC of SEQ ID NO: 2.

While the IL-1α specific Abs described above are preferred for use in the methods described herein, in some cases, other agents that specifically target IL-1α might be used so long as their administration leads to improvement of a characteristic of HS. These other agents might include vaccines that cause the production of anti-IL-1α Abs, proteins or peptides that bind IL-1α, and small organic molecules which specifically target IL-1α. Those that do not specifically bind IL-1β are preferred because the use of such agents have been reported to worsen the symptoms of HS (e.g., Tekin et al., Indian J Dermatol Venereol Leprol 2017; 83:615-7), and others have reported that IL-1β promotes healing and repair (e.g., Bersudsky et al., Gut. 2014 Apr; 63(4):598-609).

### Pharmaceutical Compositions and Methods

The anti-IL-1α Ab compositions (and other agents that specifically target IL-1α) may be administered in pharmaceutically acceptable carriers (e.g., sterile saline), that are selected on the basis of mode and route of administration and standard pharmaceutical practice. A list of pharmaceutically acceptable carriers, as well as pharmaceutical formulations, can be found in Remington’s Pharmaceutical Sciences, a standard text in this field, and in USP/NF. Other substances may be added to the compositions and other steps taken to stabilize and/or preserve the compositions, and/or to facilitate their administration to a subject.

For example, the Ab compositions might be lyophilized (see Draber et al., J. Immunol. Methods. 181:37, 1995; and PCT/US90/01383); dissolved in a solution including sodium and chloride ions; dissolved in a solution including one or more stabilizing agents such as albumin, glucose, maltose, sucrose, sorbitol, polyethylene glycol, and glycine; filtered (e.g., using a 0.45 and/or 0.2 micron filter); contacted with beta-propiolactone; and/or dissolved in a solution including a microbicide (e.g., a detergent, an organic solvent, and a mixture of a detergent and organic solvent.

In one embodiment, the pharmaceutical composition is a liquid formulation of anti-IL-1α Ab (for example, bermekimab) in a stabilizing isotonic formulation buffer at pH 6.2-6.5.

In some embodiments the pharmaceutical composition comprises the following components:
anti-IL-1α Ab (for example, bermekimab)
Trehalose Dihydrate
Sodium Phosphate Dibasic
Citric Acid Monohydrate
Water
Phosphoric Acid
Sodium Hydroxide

In some embodiments, the concentration of anti-IL-1α Ab in the pharmaceutical composition is between about 100 mg/ml and about 200 mg/ml. For instance, about 100 mg/ml, about 125 mg/ml, about 150 mg/ml, about 175 mg/ml or about 200 mg/ml.

In some embodiments, the concentration of anti-IL-1α Ab in the pharmaceutical composition is between about 100 mg/ml and about 125 mg/ml. In other embodiments, the concentration of anti-IL-1α Ab in the pharmaceutical composition is between about 125 mg/ml and about 150 mg/ml. In other embodiments, the concentration of anti-IL-1α Ab in the pharmaceutical composition is between about 150 mg/ml and about 175 mg/ml. In other embodiments, the concentration of anti-IL-1α Ab in the pharmaceutical composition is between about 175 mg/ml and about 200 mg/ml.

The Ab compositions may be administered to animals or humans by any suitable technique. Typically, such administration will be parenteral (e.g., intravenous, subcutaneous, intramuscular, or intraperitoneal introduction). In one preferred embodiment, the administration is intravenous. In another preferred embodiment, the administration is subcutaneous.

In one embodiment, the concentration of anti-IL-1α Ab (for example, bermekimab) in the pharmaceutical composition is about 100 mg/ml and the administration is intravenous.

In one embodiment, the concentration of anti-IL-1α Ab (for example, bermekimab) in the pharmaceutical composition is about 100 mg/ml and the administration is subcutaneous.

In one embodiment, the concentration of anti-IL-1α Ab (for example, bermekimab) in the pharmaceutical composition is about 150 mg/ml and the administration is subcutaneous.

In one embodiment, the concentration of anti-IL-1α Ab (for example, bermekimab) in the pharmaceutical composition is about 175 mg/ml and the administration is subcutaneous.

In one embodiment, the concentration of anti-IL-1α Ab (for example, bermekimab) in the pharmaceutical composition is about 200 mg/ml and the administration is subcutaneous.

The compositions may also be administered directly to the target site (e.g., the skin) by, for example, topical application. Other methods of delivery, e.g., liposomal delivery or diffusion from a device impregnated with the composition, are known in the art. The composition may be administered in a single bolus, multiple injections, or by continuous infusion (e.g., intravenously or by peritoneal dialysis).

A therapeutically effective amount is an amount which is capable of producing a medically desirable result in a treated animal or human. An effective amount of anti-IL-1α Ab compositions is an amount which shows clinical efficacy in patients as measured by the improvement in one or more symptoms of skin inflammation. As is well known in the medical arts, dosage for any one animal or human depends on many factors, including the subject’s size, body surface area, age, the particular composition to be administered, sex, time and route of administration, general health, and other drugs being administered concurrently. Preferred doses range from between 1-20 mg/kg body weight (e.g., 1, 2, 3, 4, 5, 6, 7, 8, 9, 10, 11, 12, 13, 14, 15, 16, 17, 18, 19, or 20 +/- 0.1, 0.2, 0.3, 0.4, 0.5, 0.6, 0.7, 0.8, or 0.9 mg/kg body weight).

In one embodiment, the anti-IL-1α Ab (for example, bermekimab) is administered at a dose of about 7.5 mg/kg body weight of the subject. In certain such embodiments, the administration is intravenous.

In some cases, a single dose is effective at resolving an episode of skin inflammation. In other cases, doses may be given repeatedly, e.g., semi-weekly, weekly, bi-weekly, tri-weekly, semi-monthly, once every three weeks, monthly, bi-monthly, or as needed (if lesions recur).

Doses may also be defined according to the amount of anti-IL-1α Ab (for example, bermekimab) that is present in the pharmaceutical composition. In some embodiment, the dose of the anti-IL-1α Ab (for example, bermekimab) is at least 200 mg (e.g., 200, 300, 350 400, 500, 600, 700, 800 or 1050 mg).

In one embodiment, the dose of the anti-IL-1α Ab (for example, bermekimab) is about 400 mg. In another embodiment, the dose of the anti-IL-1α Ab (for example, bermekimab) is about 200 mg. In a further embodiment the dose of the anti-IL-1α Ab (for example, bermekimab) is about 800 mg. In another embodiment, the dose of the anti-IL-1α Ab (for example, bermekimab) is about 1,200 mg. In another embodiment, the dose of the anti-IL-1α Ab (for example, bermekimab) is about 350 mg. In another embodiment, the dose of the anti-IL-1α Ab (for example, bermekimab) is about 700 mg. In another embodiment, the dose of the anti-IL-1α Ab (for example, bermekimab) is about 1,050 mg.

In a preferred embodiment, the dose of the anti-IL-1α Ab (for example, bermekimab) is about 400 mg and the administration is subcutaneous. In certain such embodiments, the concentration of anti-IL-1α Ab (for example, bermekimab) is about 100 mg/ml, about 150 mg/ml, about 175 mg/ml or about 200 mg/ml.

In another embodiment, the dose of the anti-IL-1α Ab (for example, bermekimab) is about 200 mg and the administration is subcutaneous. In certain such embodiments, the concentration of anti-IL-1α Ab (for example, bermekimab) is about 175 mg/ml.

In another embodiment, the dose of the anti-IL-1α Ab (for example, bermekimab) is about 800 mg and the administration is subcutaneous. In certain such embodiments, the concentration of anti-IL-1α Ab (for example, bermekimab) is about 175 mg/ml.

In another preferred embodiment, the dose of the anti-IL-1α Ab (for example, bermekimab) is about 400 mg and the administration is intravenous. In certain such embodiments, the concentration of anti-IL-1α Ab (for example, bermekimab) is about 100 mg/ml.

In another embodiment, the dose of the anti-IL-1α Ab (for example, bermekimab) is about 800 mg and the administration is intravenous. In certain such embodiments, the concentration of anti-IL-1α Ab (for example, bermekimab) is about 100 mg/ml.

In another embodiment, the dose of the anti-IL-1α Ab (for example, bermekimab) is about 1,200 mg and the administration is intravenous. In certain such embodiments, the concentration of anti-IL-1α Ab (for example, bermekimab) is about 100 mg/ml.

In another embodiment, the dose of the anti-IL-1α Ab (for example, bermekimab) is about 350 mg and the administration is subcutaneous. In certain such embodiments, the concentration of anti-IL-1α Ab (for example, bermekimab) is about 175 mg/ml.

In another embodiment, the dose of the anti-IL-1α Ab (for example, bermekimab) is about 700 mg and the administration is subcutaneous. In certain such embodiments, the concentration of anti-IL-1α Ab (for example, bermekimab) is about 175 mg/ml.

In another embodiment, the dose of the anti-IL-1α Ab (for example, bermekimab) is about 1,050 mg and the administration is subcutaneous. In certain such embodiments, the concentration of anti-IL-1α Ab (for example, bermekimab) is about 175 mg/ml.

### Combination Treatment

HS patients treated with an agent that selectively binds IL-1α can also be administered other agents. For example, such patients can be treated with corticosteroids, retinoids, resorcinol, hormones, and biologics such as adalimumab or infliximab. Antimicrobials might also be used. In particular, antibiotics or other agents that target S. aureus can be used in those patients having or suspected of having S. aureus colonization or infection in one or more HS lesions. The use of antibodies that opsonize S. aureus are believed to be particularly useful. Preferred anti- S. aureus for this use are those having Fab region paratopes that specifically bind to S. aureus protein A (SpA) and Fc regions that do not bind SpA such that there are capable of mediating opsinization of S. aureus bacteria despite S. aureus’s expression of antibody-neutralizing SpA. These are described in U.S. Pat. No. 9,416,172 (e.g., the antibody designated PA8-G3 therein).

### EXAMPLES

Example 1 - A double-blind, randomized, placebo-controlled clinical trial of the safety and efficacy of MABp1, a True Human™ antibody targeting interleukin-1α, in patients with HS.

HS patients were screened from those who are currently under follow-up. Inclusion criteria were: written informed consent provided by the patient; age 18 years or older; diagnosis of HS; HS of Hurley II or III stage disease or rapidly progressive HS of Hurley I stage; presence of 3 or more inflamed nodules consistent with HS in the body; at least one of the following: a) previous failure of treatment with any anti-TNFα, regimen; b) previous relapse under treatment with any anti-TNFα, regimen; or c) unwillingness to receive subcutaneous adalimumab treatment.

Exclusion criteria were: history of systemic lupus erythematosus, of rheumatoid arthritis or of seronegative inflammatory arthritis; treatment with any biologicals or investigational agents within the last 4 weeks (or 5 half-lives, whichever is longer); history of severe allergic or anaphylactic reactions to human, humanized, chimeric, or murine monoclonal antibodies; administration of any live (attenuated) vaccine over the last 4 weeks; history of recurrent vein thrombosis or embolism compatible with anti-cardiolipin syndrome; any present serious bacterial infection namely pneumonia, endocarditis, acute pyelonephritis and intraabdominal infection; hepatic dysfunction defined as any value of transaminases, of γ-glutamyl transpeptidase or of bilirubin> 2 x upper normal limit; history of hematological or solid tumor malignancy, arterial hypertension, liver cirrhosis, HIV infection, and hepatitis virus B or C infection; history of episodes mimicking demyelinating disorders or a definite diagnosis of multiple sclerosis; any creatinine value above 1.5 mg/dl; intake of corticosteroids defined as daily intake of prednisone or equivalent more than 1 mg/kg for the last three weeks; neutropenia defined as <1000 neutrophils/mm3; pregnancy or lactation; history of tuberculosis (latent or active); major surgery within 28 days prior to Day 0.

Diagnosis of HS was based on the following criteria, set by the 2nd Conference of the HS foundation in San Francisco: disease onset after puberty; involvement of at least two areas of the skin rich in apocrine glands; and history of recurrent painful boils without/with drainage of pus from the affected areas. Once a patient was considered eligible for the study the following procedures were performed: thorough study of record-history and medications; thorough physical examination; skin tuberculin test (any diameter below 5 mm is considered negative); chest X-ray; serology for human immunodeficiency virus (HIV), for hepatitis B virus (HBV) and for hepatitis C virus (HCV); serum creatinine; and liver biochemistry. Only patients within normal were enrolled in the study. Patients were randomly 1:1 assigned to receive either placebo or MABp1 (XBiotech USA, Inc.) intravenously. The randomization sequence was built by an independent biostatistician. The investigational drug or matched placebo was administered intravenously with a one-hour infusion every 14 days (+/- 1 day) for 12 weeks, i.e., at week 0 (baseline), week 2, week 4, week 6, week 8, week 10 and week 12 for a maximum of seven infusions. The dose of MABp1 was 7.5 mg/kg.

XILONIX™, is a sterile injectable liquid formulation of 50 mg/mL MABp1 in a stabilizing isotonic buffer (pH 6.4). Each 10-mL serum vial contains 6 ml of the formulation, and is sealed with a 20-mm grey bromobutyl stopper and flip-off aluminum seal. Product was stored at 2-8° C., with excursions to room temperature permitted. The exact composition of the drug product is shown below:

Composition of the Final Drug Product

| | | | |
|---|---|---|---|
| Ingredient | Grade | Manufacturer | Concentration |
| MABp1 antibody | GMP | XBiotech | 50 mg/ml |
| sodium phosphate dibasic | compendial | JT Baker | 12 mg/ml |
| citric acid monohydrate | compendial | JT Baker | 2 mg/ml |
| Trehalose•2H₂O (high-purity low endotoxin) | compendial | Ferro-Pfanstiehl | 60 mg/ml |
| polysorbate 80 | compendial | JT Baker | 0.2 mg/ml |
| Phosphoric acid, to adjust pH | compendial | JT Baker | 0.04 mg/ml |
| water for injection | compendial | Microbix | - - |

The placebo product was manufactured following the same procedures and batch records used to manufacture the MABp1 drug product. The placebo dosage form is a sterile isotonic formulation buffer at pH 6.2-6.5. Each 10-ml Type I borosilicate glass serum vial contains 6 mL of the formulation buffer, and is sealed with a 20-mm Daikyo Flurotec butyl rubber stopper and flip-off aluminum seal. The product was stored upright at 2-8° C., with excursions to room temperature permitted. The exact composition of the Placebo Product is shown in the table below:

Composition of Placebo Product

| | | | |
|---|---|---|---|
| Ingredient | Grade | Manufacturer | Concentration |
| trehalose dihydrate | compendial | Ferro-Pfanstiehl (USA) | 60 mg/ml |
| sodium phosphate dibasic | compendial | JT Baker (USA) | 12 mg/ml |
| citric acid monohydrate | compendial | JT Baker (USA) | 2 mg/ml |
| Polysorbate 80 | compendial | JT Baker (USA) | 0.2 mg/ml |
| Phosphoric acid, to adjust pH | compendial | JT Baker | 0.04 mg/ml |
| water for injection | compendial | Irvine Scientific (USA) | q.s. |

XILONIX™ was diluted in a 100-mL bag of normal saline prior to infusion. The following calculations were used to determine the volume of drug product to be diluted for each study subject:
50 mg/ml drug product, 7.5 mg/kg dose:
$$\begin{matrix}\text{Volume of drug product to be diluted =} \\ \text{Vd =} \frac{\left(\text{Body Weight} \times \text{Dosage}\right)}{50 \text{mg} / \text{mL}}\end{matrix}$$
(Body Weight was rounded to the nearest whole number)
$$\text{Example for 70 kg Subject at 7} \text{.5} \text{mg} / \text{kg:} \text{Vd} \text{=} \frac{\left(70 \text{kg} \times \text{7} \text{.5} \text{mg} / \text{kg}\right)}{50 \text{mg} / \text{mL}}$$
Vd = 10.5 mL (round to one decimal place)

The calculated volume (Vd) was withdrawn from the subject’s assigned vial(s) using a suitable syringe. The same amount of saline as the calculated drug was removed from the 100-ml bag. The calculated volume was then injected into the 100-mL IV bag of normal saline (0.9% NaCl), resulting in a final total volume of 100 ml. The drug product was then mixed by gently inverting the bag ten times. After priming the infusion set lines, the delivery pump was programmed to deliver 100 mL of the diluted drug product over a 1-hour period (60 +/- 15 minutes), with the subject being monitored for signs of an infusion reaction. Patients’ visits occurred at week 0, at week 2, at week 4, at week 6, at week 8, at week 10, at week 12, at week 16, at week 20 and at week 24. At every visit the following procedures were performed.

| Visits | 1 | 2 | 3 | 4 | 5 | 6 | 7 | 8 | 9 | 10 |
|---|---|---|---|---|---|---|---|---|---|---|
| Weeks | 0 | 2 | 4 | 6 | 8 | 10 | 12 | 16 | 20 | 24 |
| DQLI | X | - | - | | - | - | X | - | - | X |
| Physical examination | X | X | X | X | X | X | X | X | X | X |
| HiSCR | X | X | X | X | X | X | X | X | X | X |
| PGA | X | X | X | X | X | X | X | X | X | X |
| Disease activity | X | X | X | X | X | X | X | X | X | X |
| Modified Sartorius | X | X | X | X | X | X | X | X | X | X |
| VAS for disease | X | X | X | X | X | X | X | X | X | X |
| VAS for pain | X | X | X | X | X | X | X | X | X | X |
| Photo | X | - | - | - | - | - | X | - | - | X |
| Blood sampling | X | - | - | - | -- | - | X | - | - | X |

DQLI: Dermatology Quality of Life Index
HiSCR: Hidradenitis Suppurativa Clinical Response score
PGA: Physicians’ Global Assessment
VAS: Visual Analogue Scale

Patients were asked to provide an assessment of the severity of their disease using the visual analogue scale (VAS) in mm. They were told that 0 represents no disease activity and 100 the worst disease activity they ever felt. Patients were asked to provide one score for their overall impression about their disease and another score about the physical pain they feel. The investigators asked the patient to provide the frequency of the exacerbation of his disease and the pain felt at the affected sites. Patients were given the below DLQI score and they were asked to fill it out only at week 0, at week 12 and at week 24.

The Dermatology Quality of Life Index (DQLI). Each question is scored from 0 (absence) to 3 (intense problem)

| | Question | Score |
|---|---|---|
| 1. | How itchy, sore, painful or stinging has your skin condition been? | |
| 2. | How embarrassed or self-conscious have you been because of your skin? | |
| 3. | How much has your skin interfered with you going shopping or looking after your home or garden? | |
| 4. | How much has your skin influenced the clothes you wear? | |
| 5. | How much has your skin affected your social or leisure activities? | |
| 6. | How much has your skin made it difficult for you to do any sport? | |
| 7. | Has your skin prevented you from working or studying? | |
| 8. | How much has your skin created problems with your partner or any of your close friends or relatives? | |
| 9. | How much has your skin caused any sexual difficulties? | |
| 10. | How much of a problem has the treatment for your skin been? | |

The investigators counted the following from each individually affected area and took a photo of that area: the number of fistulas; the number of nodules or abscesses; the number of scars; their impression about the degree of inflammation scored from 0 to 3 as follows: 0, absent, 1, mild; 2, moderate; 3, intense; the two largest dimensions of each lesion in mm. Based on the above the following two scores were assessed at each visit: Hidradenitis Suppurativa Clinical Response (HiSCR) score and Physicians’ Global Assessment (PGA) score. For HiSCR, patients were defined as achievers or non-achievers. The probability of achieving a positive HiSCR score was starting from the second visit and it was defined as a ≥ 50% reduction in inflammatory lesion count (sum of abscesses and inflammatory nodules), and no increase in abscesses or draining fistulas in HS when compared with baseline. For PGA, this score was classified as: a) clear when the total number of abscesses is 0, the total number of draining fistulas is 0, the total number of inflammatory nodules is 0 and the total number of non-inflammatory nodules is 0; b) minimal when the total number of abscesses is 0, the total number of draining fistulas is 0, the total number of inflammatory nodules is 0 and there is presence of non-inflammatory nodules; c) mild when the total number of abscesses is 0, the total number of draining fistulas is 0, and the total number of inflammatory nodules is 1-4 or when there is presence of one abscess or draining fistula and absence of any inflammatory nodule; d) moderate when the total number of abscesses is 0, the total number of draining fistulas is 0 and the total number of inflammatory nodules is up to 5 or when there is presence of one abscess or draining fistula and up to one inflammatory nodule; e) severe when the total number of abscesses or draining fistulas is 2-5 and the total number of inflammatory nodules is 5-10; and f) very severe when there are more than 5 abscesses or draining fistulae.

Disease activity. This is defined as the sum of scores of all affected areas of each patient. Each area was evaluated by the following formula: (multiplication of the two largest diameters in each affected area in mm) x (the degree of inflammation of each lesion).

The modified Sartorius score. This is the sum of separate scoring for each affected area using the data recorded as follows: a) 3 points per anatomical region involved; b) 6 points for each fistula and 1 point for each nodule or abscess; c) 1 point when the longest distance between two relevant lesions in each affected area is <5 cm; 3 points when it is 5-10 cm; and 9 points when it is >10 cm; and d) 9 points when there is no clear separation of lesions from adjacent normal skin and 0 points when there is.

The efficacy of MABp1 in patients with moderate to severe HS by HiSCR scoring was assessed by the difference of achievement of positive HiSCR score between the treatment group and the comparator placebo group at week 12. The long-term efficacy of MABp1 in patients with moderate to severe HS by positive HiSCR scoring was assessed by the difference of achievement of HiSCR score between the treatment group and the comparator placebo group at week 24. Analysis was done separately for patients with previous failure or relapse under adalimumab and for patients without previous adalimumab treatment. The short-and long-term efficacy of MABp1 in patients with moderate to severe HS was assessed by the comparisons of all used scoring systems (HiSCR, PGA, DLQI, disease activity, VAS for disease, VAS for pain and modified Sartorius score) on all study visits. Analysis was also done separately for patients with previous failure or relapse under adalimumab and for patients without previous adalimumab treatment. The effect of MAbp1 on the time to new exacerbation was assessed by comparing the time to new exacerbation from week 0 between the two groups of treatment. Analysis was done separately for patients with previous failure or relapse under adalimumab and for patients without previous adalimumab treatment. Comparisons of HiSCR between the two study groups was done by the Fischer’s exact test. Comparisons of severity score for each study visits were done by non-parametric statistics. Comparison of the time to new exacerbation between the two groups was done by the log-rank test.

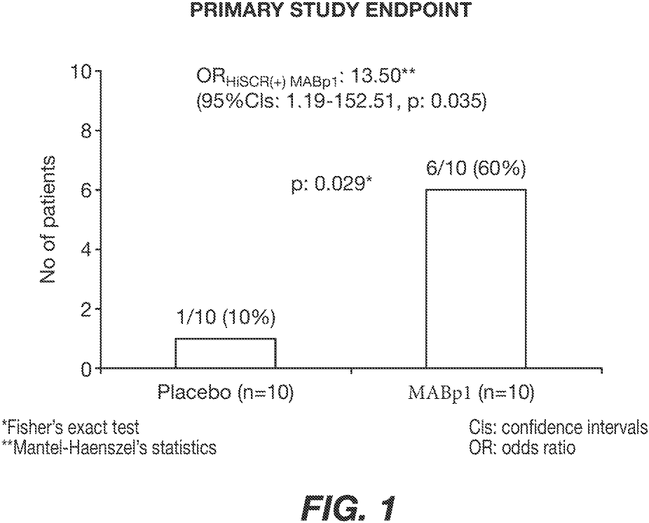

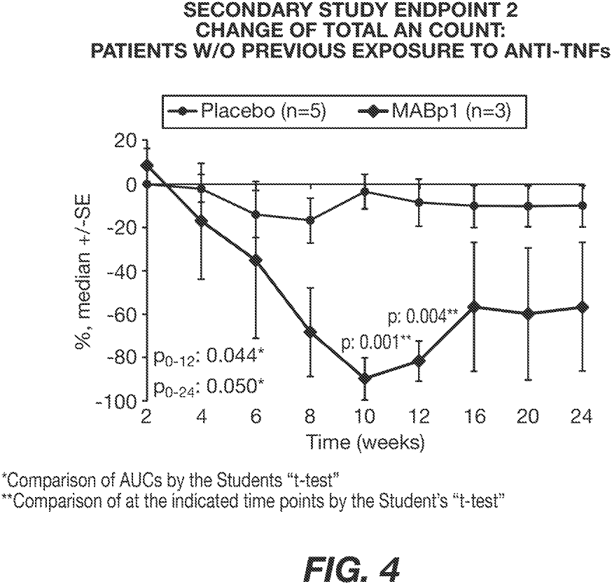

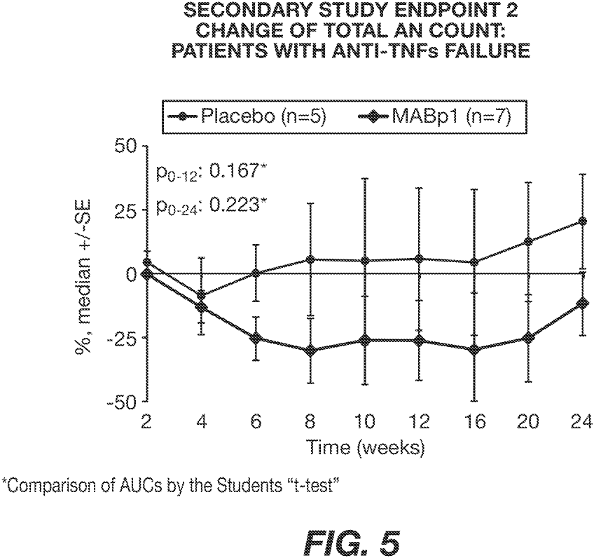

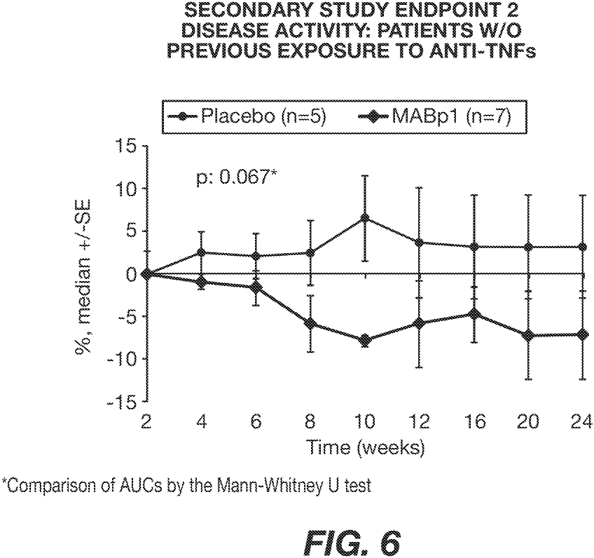

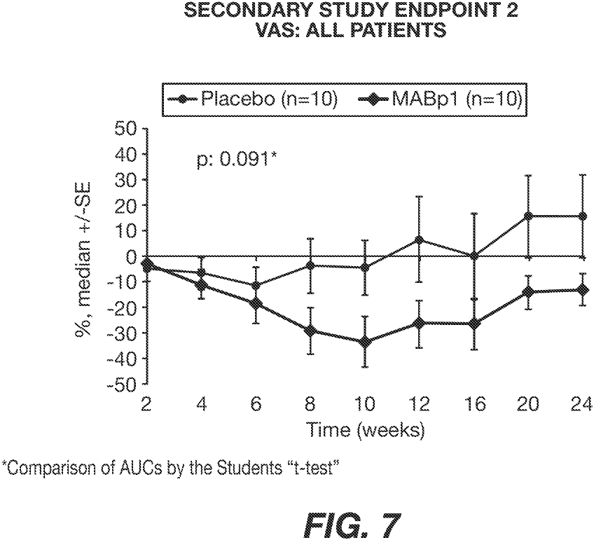

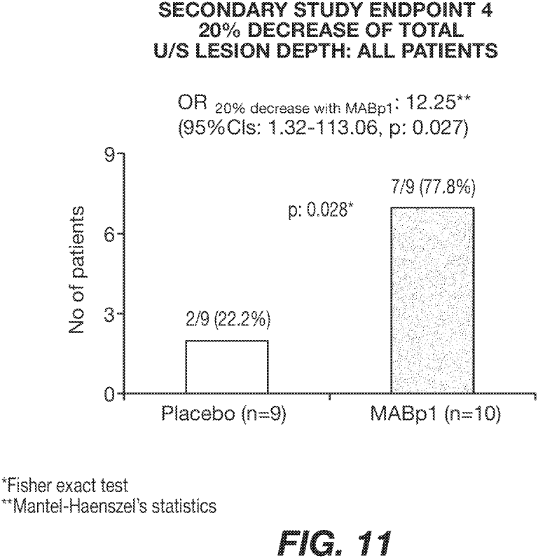

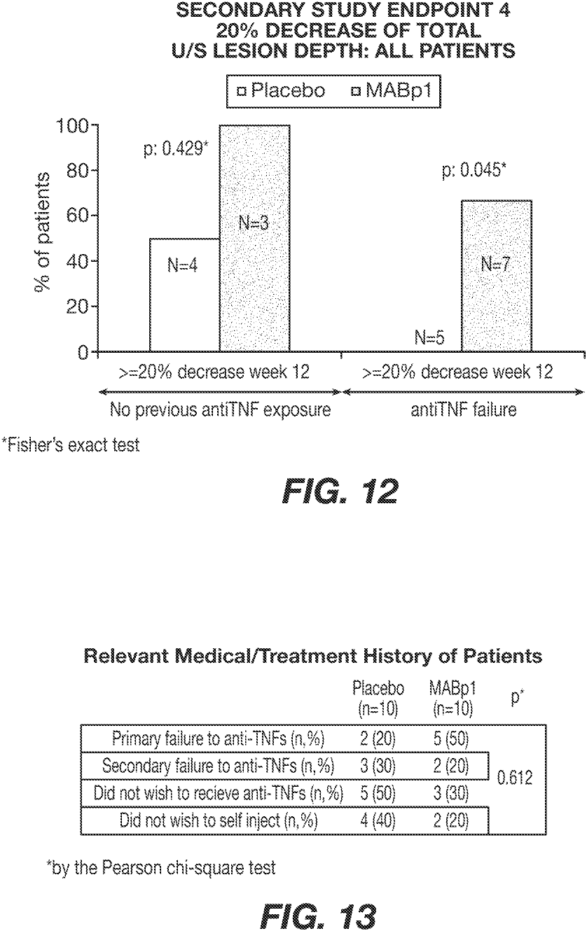

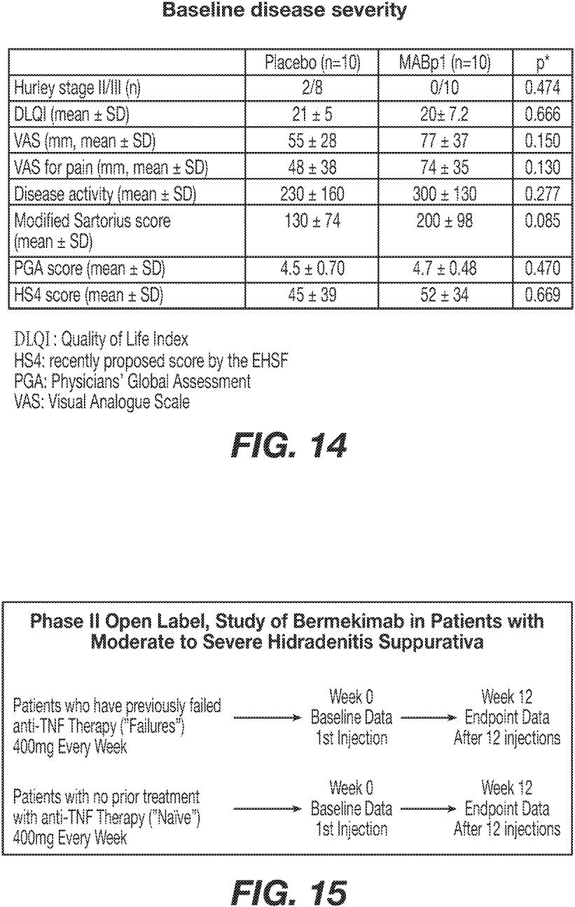

Results. FIGS. 1-12 show the results of the study. Patients treated with MABp1 achieved a significantly greater rate of positive HiSCR scores than comparators. Treatment with MABp1 was associated with significant: increased positive HiSCR scoring at week 24; decreased total AN count (more pronounced in patients without previous anti-TNF exposure); decreased VAS for the disease; prolongation of the time to new exacerbations in patients without previous anti-TNF exposure; and significant decrease of US depth of total body lesions (more pronounced in patients without previous anti-TNF failure).

The topline results from an investigator sponsored randomized Phase 2 study evaluating MABp1 as a treatment for Hidradenitis Suppurativa (HS) showed that study met its primary endpoint, demonstrating significant improvement of HS patients compared to control after 12 weeks of therapy (response rate of 60% vs 10%, respectively (p=0.035)).

The 20 patient double-blind, placebo-controlled study was designed to evaluate the safety and efficacy of MABp1, a True Human™ antibody targeting interleukin-1 alpha (IL-1α), in patients with HS not eligible for anti-TNFα therapy. Patients were randomized 1:1 to receive either MABp1 or placebo every 2 weeks for 12 weeks. Patients in the study underwent primary assessment of efficacy using Hidradenitis Suppurativa Clinical Response (HiSCR) scores at 12 weeks, continued by a follow up phase to assess time to relapse after an additional 12 weeks without therapy. Efficacy measures include assessment of HiSCR scores, a validated method for evaluating efficacy in HS patients, as well as quality of life assessment and ultrasonographic evaluation.

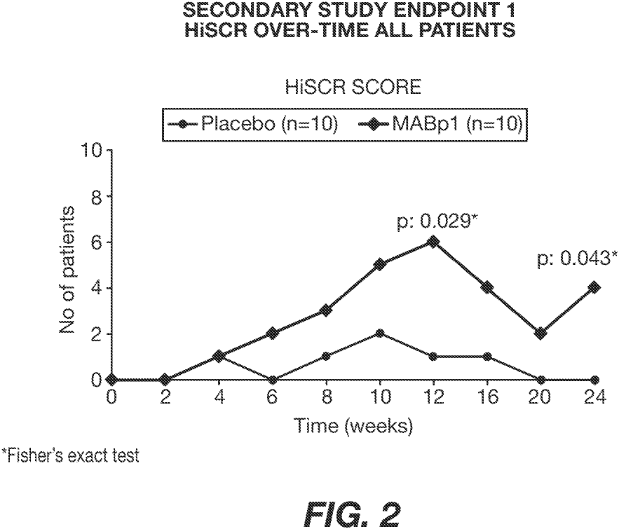

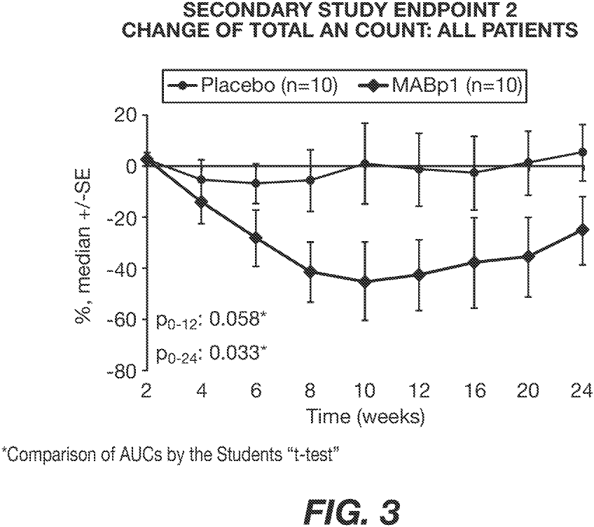

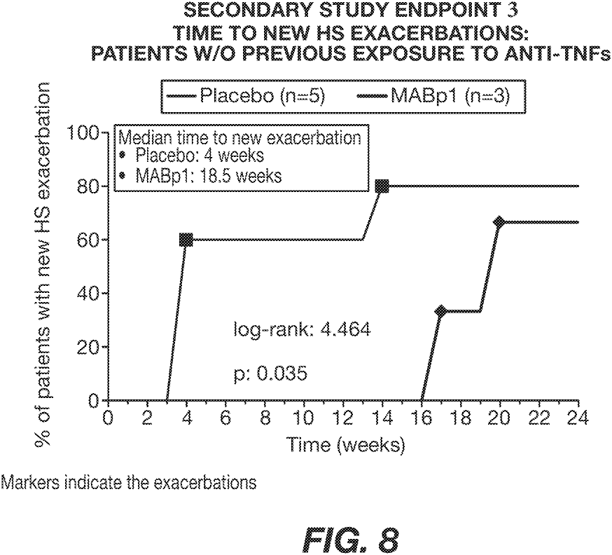

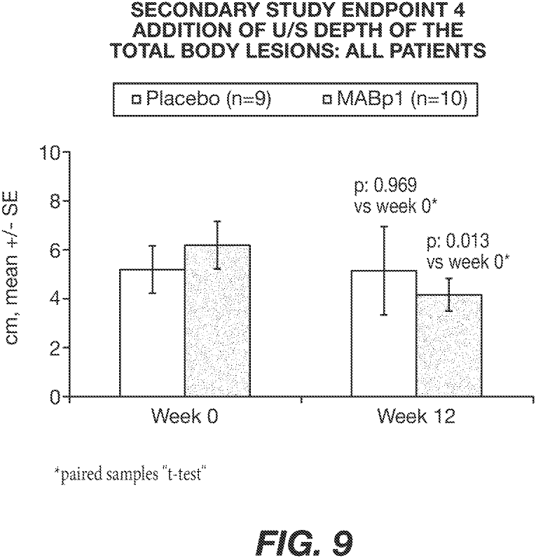

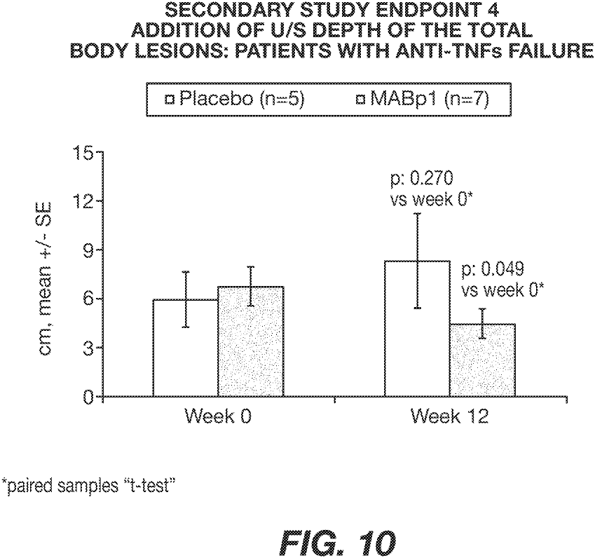

Sixty percent of patients allocated to treatment with MABp1 achieved positive HiSCR at week 12 compared to 10% of the placebo group (FIG. 1). The odds ratio (OR) for positive HiSCR under MABp1 was 13.50 (95% confidence intervals: 1.19-152.51; p= 0.035). The total AN count which is the basic component of the HiSCR score was decreased over the first 12 weeks under treatment (FIG. 3). The clinical efficacy of MABp1 was maintained until week 24, i.e., 12 weeks after treatment was stopped. At that time point, as shown in FIG. 2, no patients treated with placebo had a positive HiSCR score (0%) compared to four out of 10 patients (40%) treated with MABp1. Treatment with MABp1 was also accompanied by better patient-reported outcomes. Decrease of the visual analogue scale (VAS) was found in 30% (three out of 10) and in 70% (seven out of 10) allocated to placebo and MABp1 respectively. Sub-analysis showed that this was 40% (two out of five) and 33.3% (one out of three) respectively among anti-TNFs naïve patients and 20% (one out of five) and 85.7% (six out of seven) among patients failing previous treatment with anti-TNFs. The median time to the first HS exacerbation was seven weeks in the placebo group and 11 weeks in the MABp1 group. This time did not differ significantly between groups (log-rank: 1.98, p= 0.159). However, when sub-analysis was done among anti-TNFs naive patients, it was found that the median time until a new HS exacerbation was 4 weeks with placebo treatment and 18.5 weeks with MABp1 treatment (log-rank test: 4.46; p= 0.035; see FIG. 8). A decrease in disease activity was found in all patients treated with MABp1 and who achieved positive HiSCR at weeks 12 and 24. A decrease of at least two of the assessed scores i.e. Physicians’ Global Assessment (PGA), disease activity, modified Sartorius score, VAS for pain, and dermatology life quality index (DLQI) at week 12 was found in 40% of patients allocated to placebo and 80% of patients allocated to MABp1 (80%) (OR= 14.50; 95% confidence intervals: 0.96-218.99; p= 0.054). Sub-analysis showed that this was 60% (three out of five) and 100% (three out of three) respectively among anti-TNFs naive patients and 20% (one out of five) and 71.4% (five out of seven) among patients failing previous treatment with anti-TNFs. Significant changes in variables for skin ultrasound included total lesion vascularity and total lesion depth, which is the sum of the grading of vascularity and the sum of the greatest depth of all involved skin areas, respectively. Both variables were decreased after treatment with MABp1 (FIGS. 9-12). More than 20% decrease of total lesion depth was selected as a cut-off point, and it was found in 22.2% of patients allocated to placebo compared to 77.8% of patients treated with MABp1 (OR= 12.25; 95% confidence intervals 1.33-113.06; p= 0.027). The effect was pronounced among patients who have failed previous anti-TNFs (FIG. 10). Significant improvement in the elasticity of the affected areas was also noted.

Serum IL-1α was below the lower limit of detection in the sera sampled from all patients both before and at the end of blind treatment. Pus was sampled before treatment from six patients allocated to placebo and seven patients allocated to MABp1. Mean ± SE concentrations of IL-1α were 697.2 ± 440.4 pg/ml and 772.0 ± 221.7 pg/ml respectively (p= 0.412 by the Mann-Whitney U test). Treatment with MABp1 was accompanied by decrease of serum IL-8. More than 30% decrease of IL-8 on week 12 was selected as a cut-off point. The OR for this cut-off point by MABp1 was 13.50 (95% confidence intervals: 1.19-152.51; p= 0.035). This was consistent with change in levels of IL-8 produced from whole blood stimulated with heat-killed Staphylococcus aureus, which was significantly lower among patients treated with MABp1 than patients treated with placebo. The capacities of whole blood to produce both IL-1α and human β-defensin (hBD)-2 were positively associated among placebo-treated patients. Among the same patients, the capacity for hBD-2 production was negatively correlated with the change of the skin depth of the lesions at ultrasound. These correlations ceased to exist among MABp1-treated patients, which suggested an hBD-2-associated mode of action of MABp1 in HS that was mediated through the inhibition of IL-1α.

Safety - no study drug related adverse events or serious adverse events occurred in the study.

Analysis of the data using the iHS4 score for all 20 patients who were randomized to receive either placebo or MABp1 therapy in the Phase 2 double-blind study was performed. At least a 30% decrease of the iHS4 score from the baseline at week 12 was associated with 100% sensitivity for positive HiSCR score (the efficacy measure used in the phase 2 study). This change was found in one (10%) and in four (40%) patients allocated to placebo and MABp1, respectively (p= 0.046).

Patients that had originally been allocated to placebo in the Phase 2 study were allowed to receive treatment with the MABp1 antibody therapy in a so called open label extension (OLE) study. Seven of 10 patients that had originally received placebo were treated with MABp1 for 12 weeks. Main endpoints used in the OLE included safety and HiSCR score at the end of the 12 week treatment. At the conclusion of the double-blinded study, only one patient (1 of 10, or 10%) receiving placebo had achieved HiSCR. During the OLE, five patients (5 of 7, or 71.4%) achieved the HiSCR response (p=0.035). There was a total of 24 HS exacerbations during the blinded portion of the study compared to just 1 exacerbation during the OLE phase.

“The overall response rate observed in the data is, in my opinion, groundbreaking for the treatment of HS,” Dr. Giamarellos-Bourboulis commented, “I am truly encouraged by these results and very much look forward to the future use of MABp1 as a treatment for this devastating condition.”

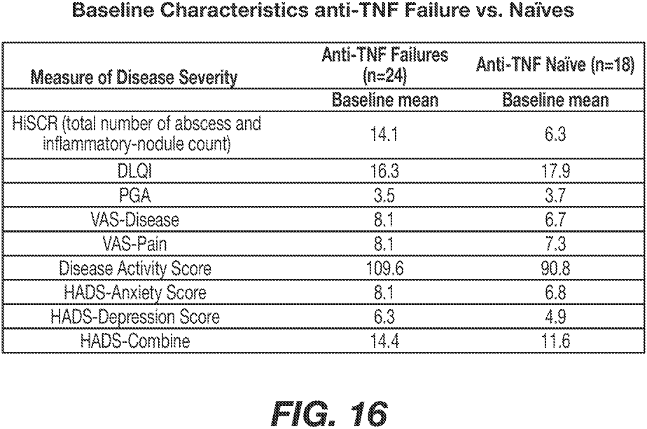

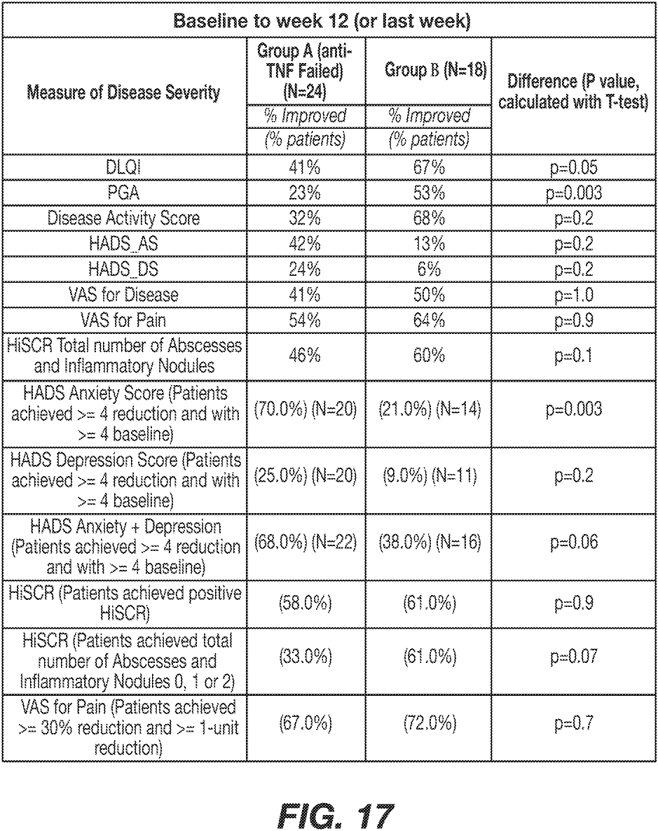

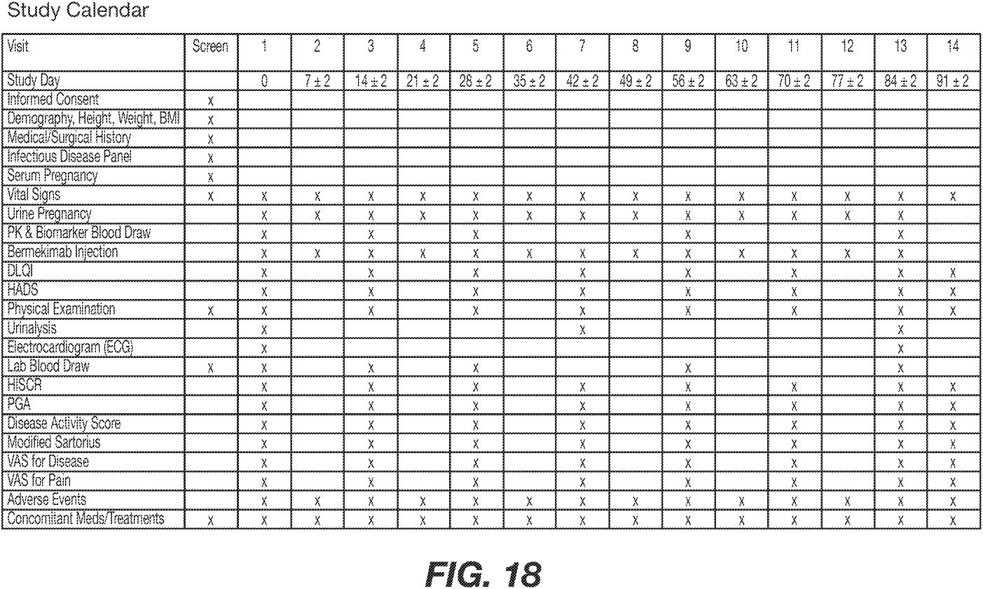

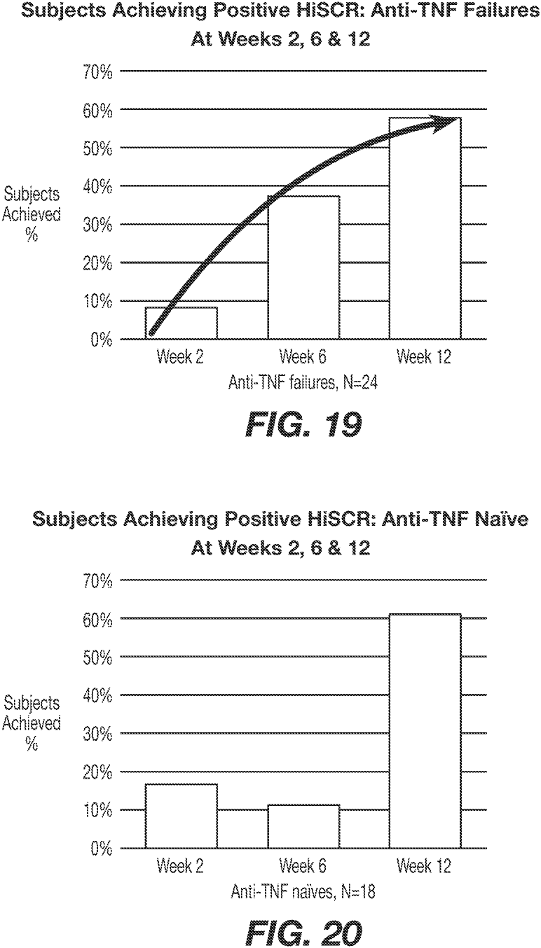

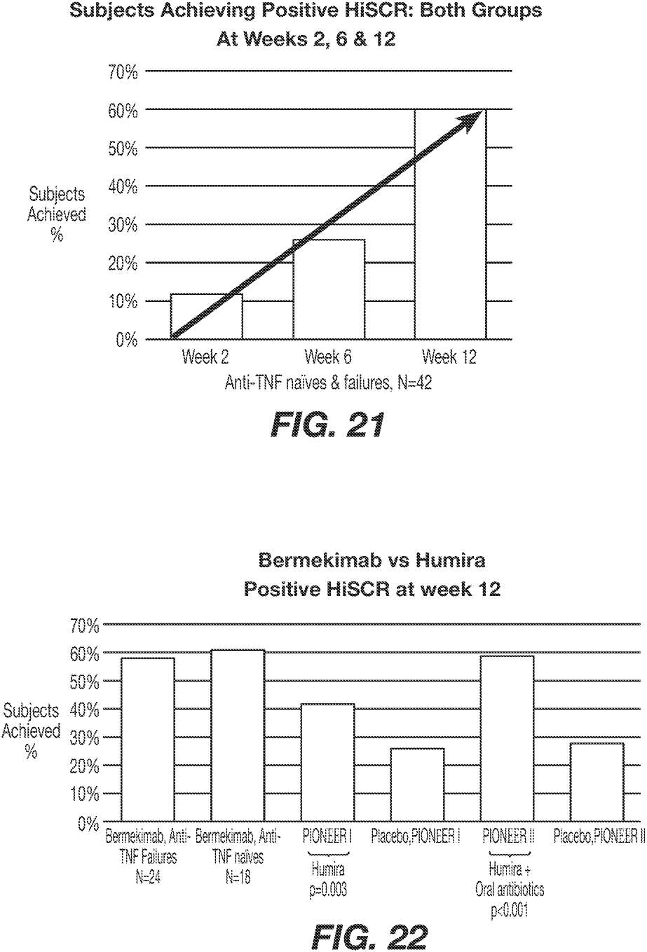

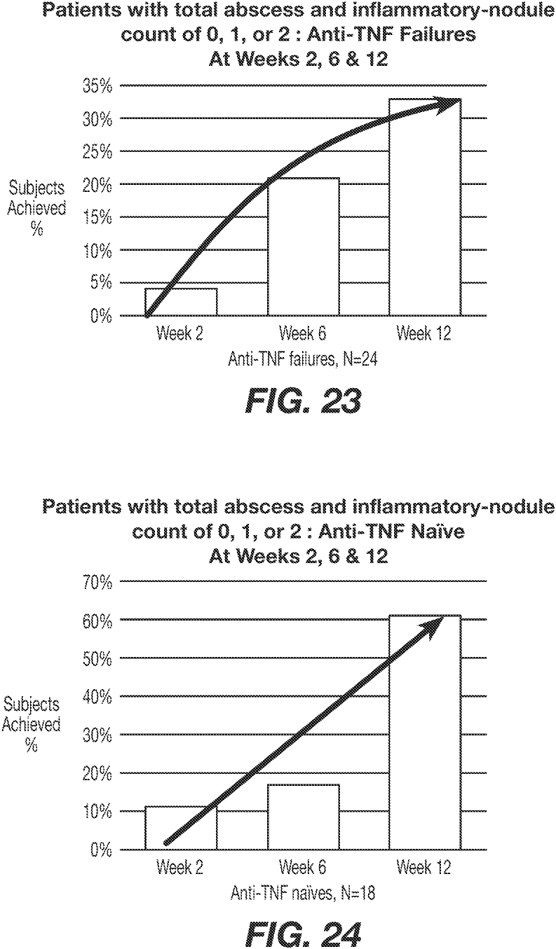

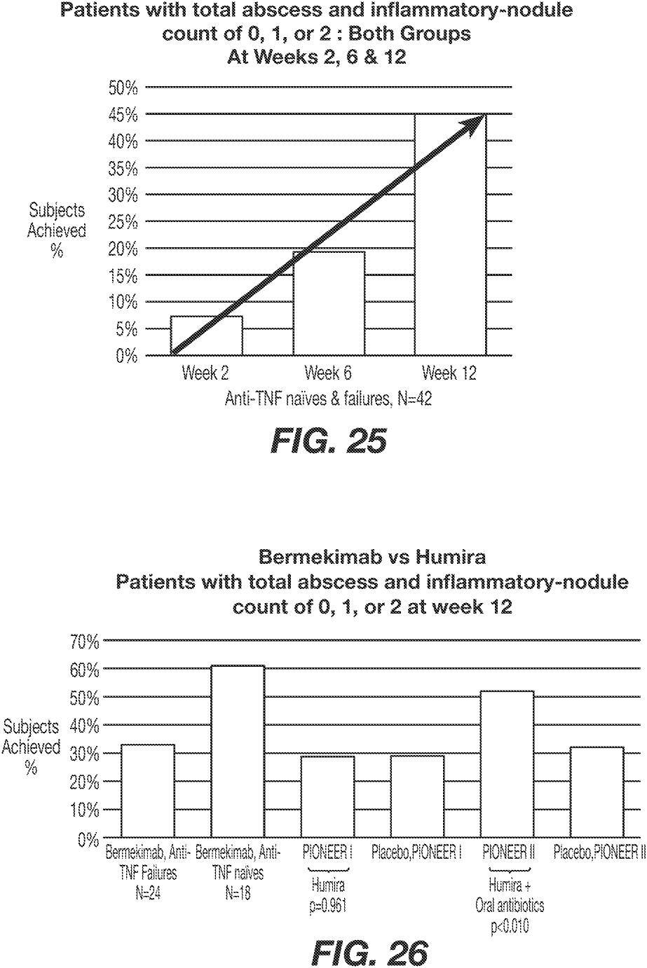

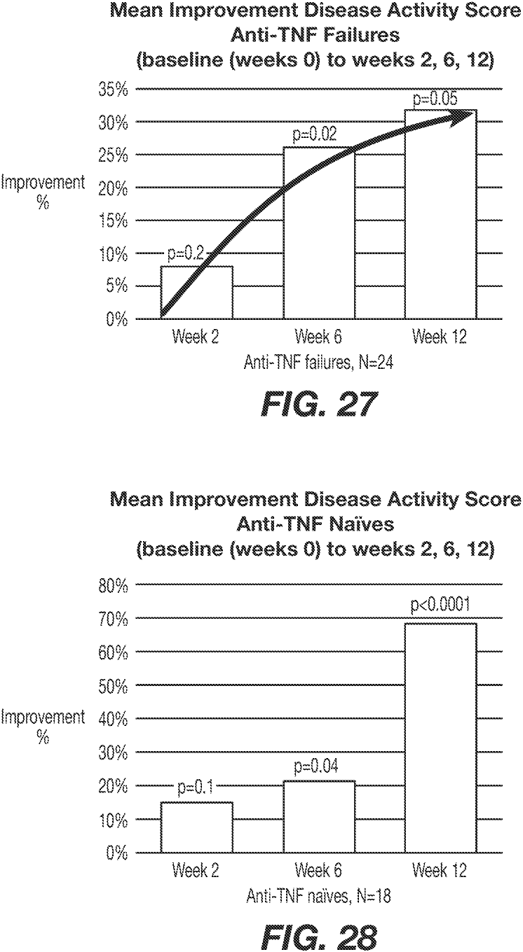

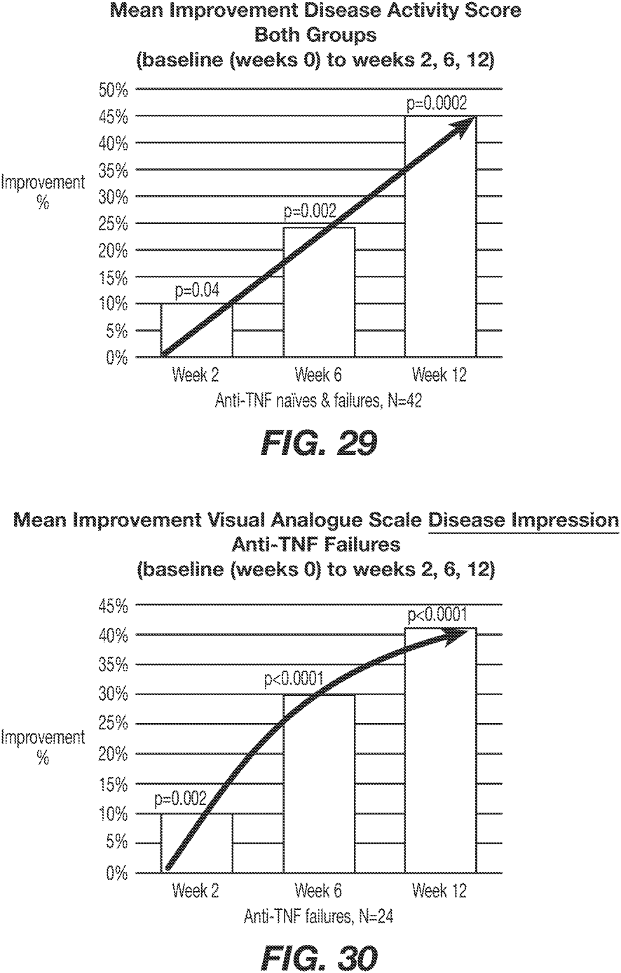

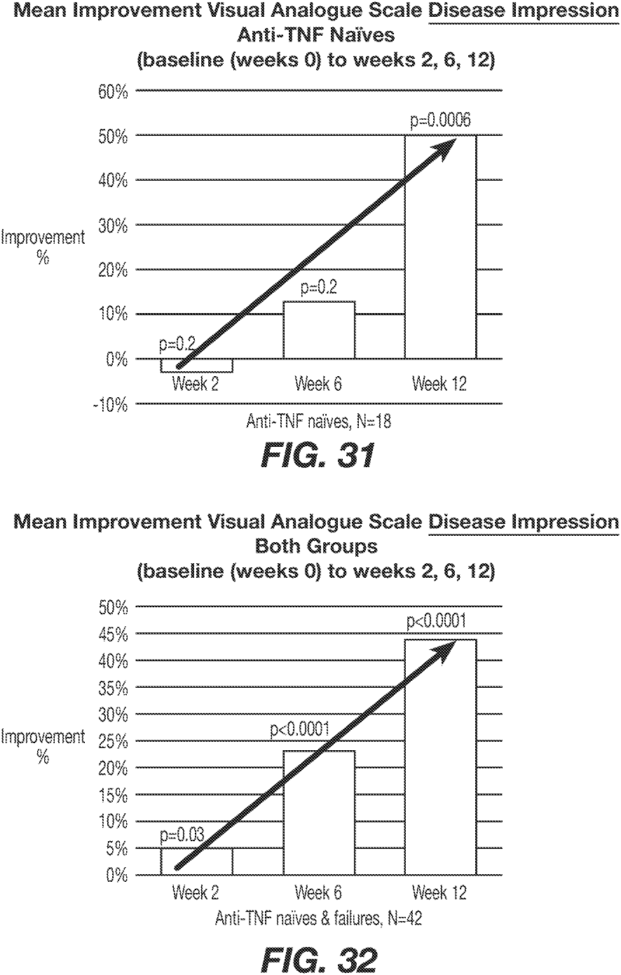

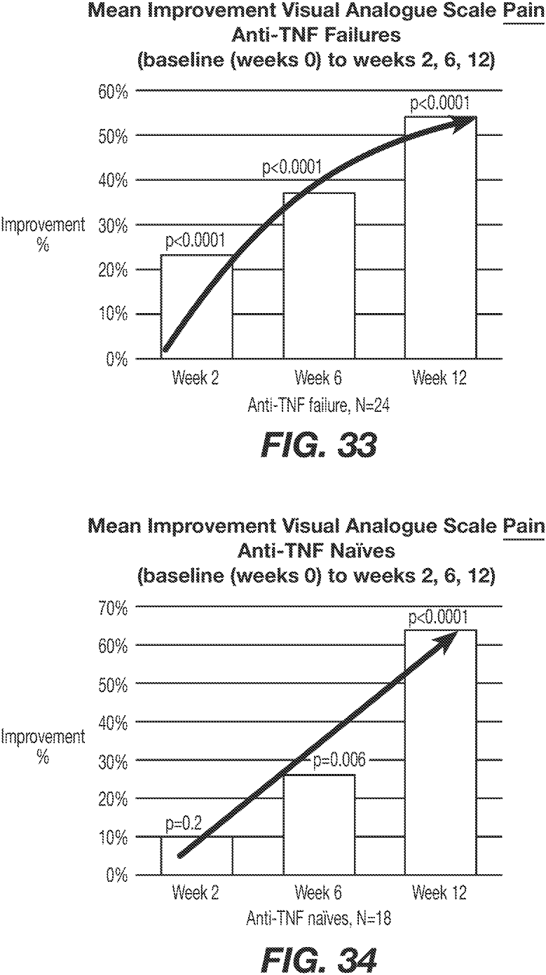

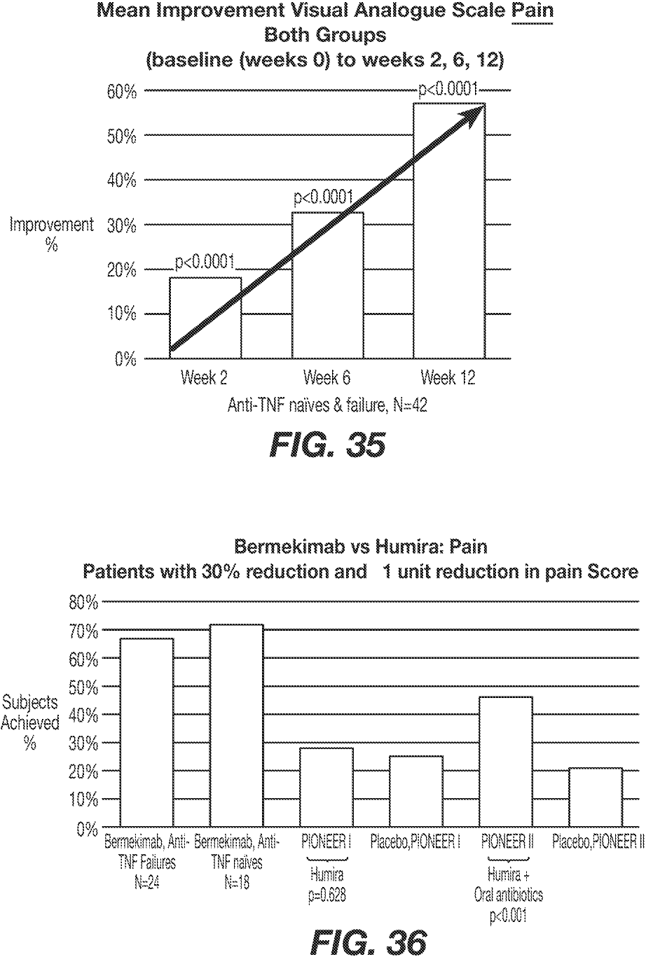

Example 2 - a Phase II Open Label, Study of Subcutaneously Administered Bermekimab (MABp1) in Patients with Moderate to Severe Hidradenitis Suppurativa. Bermekimab was formulated for subcutaneous administration at 400 mg per weekfor 12 weeks as shown in FIG. 15. The baseline characteristics of study subjects (anti-TNF failures vs. anti-TNF naives) is shown in FIG. 16. A summary of the results is presented in FIG. 17 where Group A is subjects who previously failed anti-TNF treatment and Group B is subjects never administered anti-TNF treatment. The Study calendar is shown in FIG. 18. Detailed results of the study are shown in FIGS. 19-36.

### Example 3

The objective of this study was to evaluate the safety and efficacy of bermekimab, an IL1-1α inhibitor, in the treatment of hidradenitis suppurativa (HS). This study was a phase II, multicenter, open-label study of two dose cohorts of bermekimab in patients with moderate-to-severe HS who are naive to or have failed prior anti-TNF therapy. Patients with HS (n = 42) were divided into groups A and B based on whether or not they had previously failed an anti-TNF therapy. In group A (n = 24), bermekimab was administered subcutaneously at a dose of 400 mg weekly (13 doses) in patients who had previously failed anti-TNF therapy; in group B (n = 18), bermekimab was administered subcutaneously at a dose of 400 mg weekly (13 doses) in patients who were anti- TNF naive. Bermekimab, previously found to be effective in treating HS, was evaluated using a subcutaneous formulation in patients with HS naïve to or having failed anti-TNF therapy. There were no bermekimab-related adverse events with the exception of injection site reactions. Bermekimab was effective despite treatment history, with 61% and 63% of patients naive to and having failed anti-TNF therapy, respectively, achieving HS clinical response after 12 weeks of treatment. A significant reduction in abscesses and inflammatory nodules of 60% (P < 0.004) and 46% (P < 0.001) was seen in anti-TNF naive and anti-TNF failure groups, respectively. Clinically and statistically significant reduction was seen in patients experiencing pain, with the Visual Analogue Scale pain score reducing by 64% (P < 0.001) and 54% (P < 0.001) in the anti-TNF naive and anti-TNF failure groups, respectively. IL-1α is emerging as an important clinical target for skin disease, and bermekimab may represent a new therapeutic option for treating moderate- to-severe HS.

Abbreviations: AE, adverse event; HiSCR, hidradenitis suppurativa clinical response; HS, hidradenitis suppurativa; PGA, Physician’s Global Assessment; SAE, serious adverse event; VAS, Visual Analogue Scale

The purpose of this clinical study was to evaluate the safety, tolerability, and efficacy of bermekimab in patients with moderate-to-severe HS who had either failed initial treatment with anti-TNF agents or never received anti-TNF treatment.

### Results

### Participant Flow

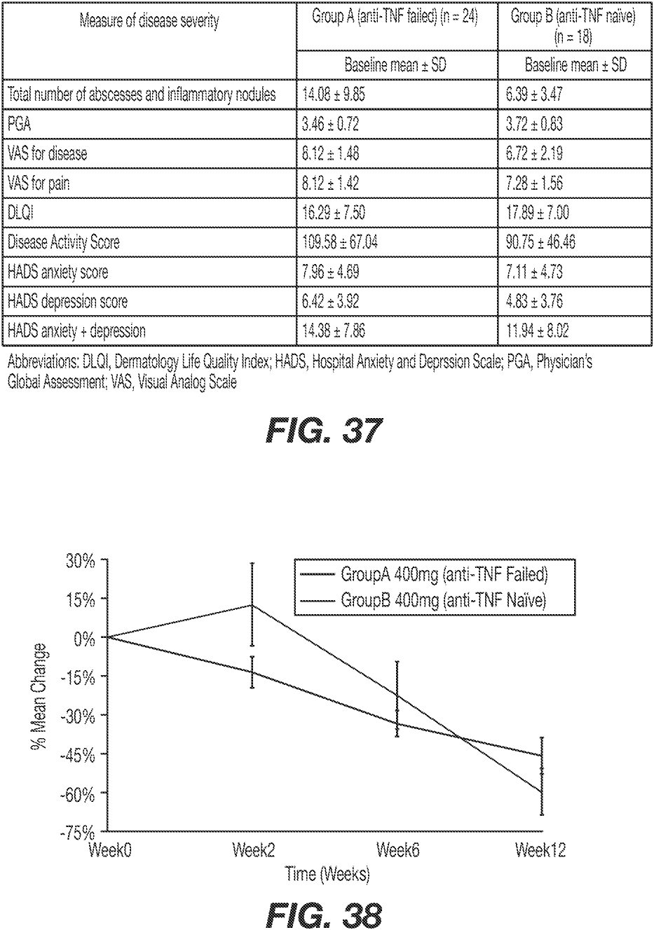

The study was conducted between July 2018 and January 2019. The trial ended at the end of follow- up for the last patient. In total, 42 subjects were enrolled into the study, 24 of whom had received and failed to respond to anti-TNF therapy (group A) and 18 of whom had not received any prior anti-TNF therapy (group B). Both groups were originally planned to receive 200 mg subcutaneous injections of bermekimab for 12 weeks; however, preliminary safety data from XBiotech’s PT044 atopic dermatitis study demonstrated good levels of safety and tolerability for 400 mg subcutaneous injections. A revised study design thus implemented 400 mg subcutaneous injections of bermekimab for 12 weeks. Baseline characteristics for enrolled patients in both groups are provided in FIG. 37.

### Primary Study Endpoint: Safety and Tolerability

The primary endpoints of this study were safety and tolerability. Bermekimab was well tolerated in all subjects throughout the study. Subcutaneous formulation of bermekimab 400 mg weekly for 13 consecutive weeks (week 0 to week 12; 13 doses) was studied in two cohorts in patients with moderate-to-severe HS, those who were naive to an anti-TNF therapy and those who had previously failed an anti-TNF therapy.

Safety was assessed by monitoring adverse events (AEs), vital signs, physical examinations, and clinical laboratory measurements. There were no discontinuations because of serious adverse events (SAEs). Overall, there were 58 non-SAEs reported, and most of them were grade I (59%) and grade II (36%). The most common AEs were injection site reactions (five grade II reactions in two subjects) and nausea (six grade II reactions in one subject). There were two SAEs in the study, (i) fall (grade III severity; not related to study drug; SAE criteria, hospitalization) and (ii) HS pain (grade III severity; not related to study drug; SAE criteria, hospitalization). All remaining AEs were mild to moderate in severity.

All clinical laboratory abnormalities were either present at screening and did not progress over the course of the study, were reflective of known underlying comorbidities, or were the result of laboratory error. Similarly, abnormalities identified during vital signs assessment were all consistent with known comorbid pathology (most commonly primary essential hypertension) and none were clinically significant. Electrocardiogram findings were not clinically significant, and no consistent pattern of abnormalities emerged in association with bermekimab.

### Secondary Study Endpoint: Clinical Efficacy

Statistically significant improvement from baseline was seen for nearly all disease severity measures in both treatment groups. The clinical efficacy of bermekimab was assessed through week 13 (or the subject’s final visit if he or she discontinued or was lost to follow-up).

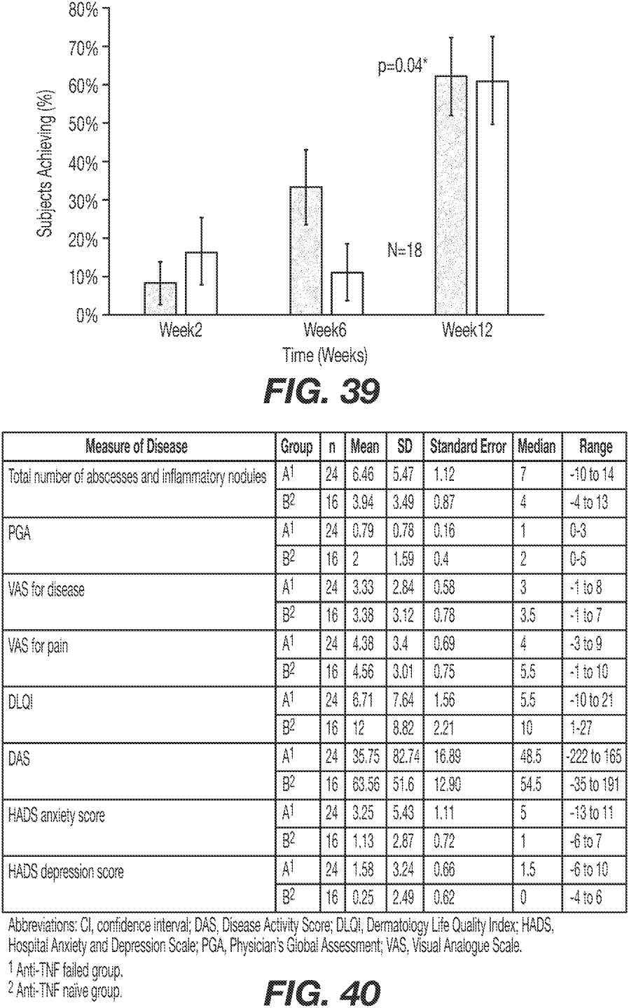

At that time point, subjects in group A (those that previously failed therapy with an anti-TNF agent) experienced an average reduction of 46% in inflammatory nodule and abscess count compared with baseline (P < 0.0001), and subjects in group B (those who were anti-TNF naive) experienced an average reduction of 60% in inflammatory nodule and abscess count compared with baseline (P = 0.004) (FIG. 38). HiSCR is used to assess disease activity in patients with HS. HiSCR is binary, in that it is either satisfied/met or not satisfied/met. To satisfy/meet HiSCR, a patient must have at least a 50% reduction in total abscess and inflammatory nodule count with no increase in abscess count and no increase in draining fistula count compared with baseline. In this study, patients were evaluated for whether or not they satisfied/met HiSCR at week 12 relative to their week 1 baselines. In groups A and B, 63% and 61% of patients, respectively, achieved HiSCR when compared with their baseline visit (FIG. 39)

Assessment of subjects’ disease activity using both the Disease Activity Score (Giamarellos- Bourboulis et al., 2008) and Physician’s Global Assessment (PGA) (Kimball et al., 2012) showed significant improvements at week 12 relative to their baseline visit. Subjects in group A showed on average a 33% reduction in Disease Activity Score at week 12 (P = 0.02), whereas subjects in group B showed an average reduction of 66% at week 12 (P = 0.001). Subject PGA scores at week 12 in group A showed a 23% average reduction (P = 0.0018), whereas subjects in group B showed a 53% average reduction in PGA score (P < 0.0001).

Treatment with bermekimab was also accompanied by better patient-reported outcomes. Improvements in the Visual Analogue Scale (VAS) for pain and disease were reported at week 12 relative to baseline in both groups. Group A showed an average improvement of 41% (P < 0.0001) and 54% (P < 0.0001) for pain and disease, respectively, whereas subjects in group B showed an average improvement of 50% (P < 0.0001) and 64% (P < 0.0001) for pain and disease, respectively.

Subjects in both groups also reported improvements in the Dermatology Life Quality Index at week 12 relative to baseline. Subjects in group A achieved an average of 41% improvement (P < 0.0001), whereas subjects in group B achieved an average of 67% improvement (P < 0.0001).

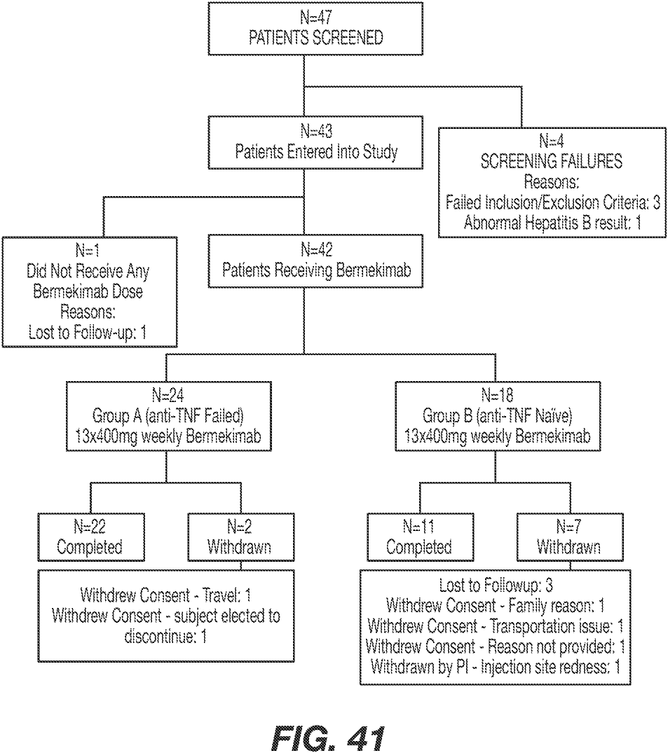

Subjects in group A also reported improvements from baseline at week 12 on the Hospital Anxiety and Depression Scale (Zigmond and Snaith, 1983). Group A subjects achieved an average of 41% improvement in the anxiety score (P = 0.001), 25% average improvement in the depression score (P = 0.02), and 34% average improvement in the overall score (P = 0.002). Although group B subjects achieved, on average, improvement in all measures of the Hospital Anxiety and Depression Scale, none of these improvements was found to be statistically significant. Descriptive statistics summarizing patient endpoint outcomes can be seen in FIG. 40.

### Discussion

HS is a debilitating inflammatory skin condition with significant disease burden. Existing treatment options are not effective for all patients or may be contraindicated for some patients. There is therefore a significant unmet need for patients with HS. Bermekimab, through its mechanism of neutralizing IL-1α, functions to combat the inflammatory cascade that leads to the phenotype seen in moderate-to-severe HS. It is a novel therapeutic that shows significant promise for HS. In this study, we demonstrate the safety, tolerability, and clinical efficacy of bermekimab in treating moderate-to-severe HS. Patients in both treatment groups evaluated in this study showed statistically significant improvement in nearly every study endpoint at 12 weeks compared with baseline.

This study importantly demonstrates the potential of bermekimab to treat patients with moderate-to- severe HS who are recalcitrant to adalimumab. Before this study, two phase III PIONEER studies allowed for adalimumab to be registered as an indicated treatment for moderate-to-severe HS. The investigators used the HiSCR score after 12 weeks as the primary efficacy outcome (Kimball et al., 2016). HiSCR achievement with adalimumab was reported in 41.8% of patients in the PIONEER I study and 58.9% of patients in the PIONEER II study (Kimball et al., 2016), which allowed antibiotic use. Adalimumab is an important advance for the treatment of HS. However, there is still a considerable unmet need for the subset of patients who failed or relapsed with adalimumab, including 41-58% of patients who have primary response failures after 12 weeks of adalimumab treatment and 30-50% of patients who have a positive initial response to treatment with adalimumab but relapse after 12 weeks of therapy. Treatment with bermekimab in patients who previously failed or relapsed after treatment with adalimumab led to HiSCR achievement by 61%. The ability of bermekimab to allow this cohort of patients to achieve such significant disease improvement is exciting and provides significant hope to many patients with HS who may feel hopeless with this debilitating disease. Bermekimab also provides hope for patients who may have contraindications to adalimumab, as evident by the significant improvements in study endpoints achieved by the cohort of patients who were naive to anti-TNF therapy.

### Materials and Methods

### Study Design

This study was a phase II, multicenter, open-label study of two dose cohorts of bermekimab in patients with moderate-to-severe HS who are naive to or have failed prior anti-TNF therapy. This trial was registered with clinicaltrials.gov (NCT03512275). The duration of subject participation was approximately 16 weeks, including a 3-week screening period and a 13 \-week treatment period. The study consisted of two groups: (i) group A (n = 24), bermekimab administered via subcutaneous injection weekly (13 doses) in patients who had previously failed anti-TNF therapy, and (ii) group B (n = 18), bermekimab administered via subcutaneous injection weekly (13 doses) in patients who were anti-TNF naive. Because there was no prior clinical data for the 400 mg weekly dosing, a power calculation between groups was not possible, and sample size was thus exploratory. Both groups were originally administered 200 mg doses of bermekimab; however, preliminary safety data from XBiotech’s PT044 atopic dermatitis study demonstrated good levels of safety and tolerability for 400 mg injections of bermekimab. The dose was subsequently increased to 400 mg in both groups A and B. A summary of subject allocation is found in FIG. 41. Patients were followed for 13 weeks to allow for assessment of safety and preliminary efficacy.

The limitations of the study design include a small sample size and uneven subject withdrawals between the two study groups. Because there was no prior clinical data for the 400 mg weekly dosing, a power calculation between groups was not possible, and sample size was thus exploratory. Because of this, although statistically significant differences were observed between the two study groups for nearly every study endpoint, these results are in the context of a limited sample size and may require additional testing with larger sample sizes in the future to confirm the efficacy of bermekimab in the population. It should be additionally mentioned that there were more withdrawals from group B (7) than group A (2). Although only one of the withdrawals was related to the drug (injection site redness), the larger difference between group numbers by the end of the study as compared with the start may have had an effect on the findings seen at study endpoint, despite the statistical significance found.

### Study Ethics, Inclusion Criteria, and Exclusion Criteria

Patients were enrolled after written informed consent. The protocol was approved by the institutional review board or ethics committee of the participating study sites. Further, the trial was conducted in compliance with the protocol, good clinical practice, and all applicable regulatory requirements.

Patients 18 or older diagnosed with HS for at least 1 year before screening with at least two distinct anatomic areas affected, one of which had to be Hurley II or III stage HS, and with a total body count of no less than three inflamed nodules and abscesses were included in the study. For group A, patients must have received and failed at least one anti-TNF therapy previously. Group B subjects must not have received any prior treatment with any anti-TNF agent. Patients who received the 200 mg dose of bermekimab in this study were eligible to begin receiving the 400 mg dose beginning at the patient’s next scheduled visit for the remainder of his or her treatment plan. Female patients of childbearing potential willing to use one method of contraception of high efficacy during the entirety of the study were included. Female patients of nonchildbearing potential were considered with a medical history that indicated that pregnancy was not a reasonable risk.

Main exclusion criteria included hepatic dysfunction, chronic infections by hepatitis B and C viruses and HIV, neutropenia, pregnancy or lactation, recent vaccination during the four weeks before screening, and history of treatment with bermekimab for any reason except patients previously treated with 200 mg in the previous versions of this study. Further, history of severe allergic or anaphylactic reactions to human, humanized, chimeric, or murine monoclonal antibodies; intake of opioid analgesics within 14 days before screening; major surgery (requiring general anesthesia or respiratory assistance) within 28 days before day 0 of start of study drug; known or suspected history of immunosuppression; and Stage C Child-Pugh liver cirrhosis were included as exclusion criteria. Patients receiving oral antibiotic treatment or systematic therapies for HS within 28 days before screening were excluded from the study as well as patients receiving topical therapies 14 days before screening.

If a patient was discontinued from the study, the reason for discontinuation was clearly documented in the source documentation and the electronic data capture. Study therapy was immediately discontinued for any the following reasons: (i) withdrawal of informed consent; (ii) any clinical AE, laboratory abnormality, intercurrent illness, or clinical progression of disease that indicates that the continued participation is not in the best interest of the subject; (iii) pregnancy; (iv) termination of the study by the sponsor; and (v) imprisonment or compulsory detention for medical treatment.

### Screening and Treatment

Once a patient was considered eligible for the study, the following was examined or performed at screening: (i) medical history and physical examination; (ii) height, weight, and body mass index; (iii) vital signs; (iv) serum creatinine and liver biochemistry; (v) complete blood count with differential and platelets;

(vi) serum pregnancy test; (vii) serology for interferon-gamma release assay, HIV, hepatitis B virus, and hepatitis C virus; and (viii) inflammatory markers C-reactive protein and erythrocyte sedimentation rate. Treatment was provided at future visits. The study drug was provided by XBiotech (Austin, TX), who built the randomization sequence. This was an open-label study.

### Follow-Up

At weeks 0 (baseline), 2, 4, 6, 8, 10, and 12 before administration of drug, patients completed the Dermatology Life Quality Index, Hospital Anxiety and Depression Scale, and a physical examination and were self-assessed for impression of their HS and pain severity using the VAS, ranging from 0 (not at all severe disease or no pain) to 10 (extremely severe disease or worst imaginable pain); individual lesions were counted and HiSCR, PGA, and disease activity were scored; and patients’ vital signs were assessed. Patients were then injected with the proper amount of bermekimab through a subcutaneous injection. Patients were monitored for 1 hour, then vital signs were reassessed 1 hour after injection. Lastly, patients were asked for any AEs and SAEs. At weeks 0, 6, and 12, in addition to the procedures described, urinalysis and an electrocardiogram were performed to further assess the safety of bermekimab.

At weeks 1, 3, 5, 7, 9, and 11, patients’ vital signs were assessed. Patients were then injected with the proper amount of bermekimab through a subcutaneous injection. Patients were monitored for 1 hour, then vital signs were reassessed 1 hour after injection. Patients were then asked for any AEs and SAEs.

At every week except for the follow-up week 13, urine pregnancy tests were collected from female subjects.

Week 13 included the following: (i) patients completed the Dermatology Life Quality Index, Hospital Anxiety and Depression Scale, and a physical examination; (ii) patients were self-assessed for HS and pain severity using the VAS from 0 (absent) to 10 (worst ever felt); (iii) individual lesions were counted and HiSCR, PGA, and disease activity were scored; and (iv) patients’ vital signs were assessed. Patients were then asked for any AEs and SAEs.

Blood draws were performed at screening and at weeks 2, 4, 8, and 12 for serum creatinine and liver biochemistry, complete blood count with differential and platelets, and pharmacokinetics/biomarker analysis. An ELISA was developed to specifically measure bermekimab levels in human plasma. Blood was drawn into a single 6-ml collection tube at each pharmacokinetics collection time point (samples were collected before dosing at the investigative site per the study lab manual and immediately shipped to the sponsor for pharmacokinetics analysis).

Any AE of grade II or higher was required to be entered into the electronic case report form within 24 hours of learning of the event. Any grade III or greater injection site reaction was to be reported to the sponsor within 24 hours of learning of the event. All SAEs were reported to the sponsor within 24 hours of knowledge of the event. These immediate reports were followed promptly by detailed, written reports. The subject was followed up with until stabilization of the reported SAE, either with full satisfactory resolution or resolution with sequelae or until death of the subject. Before declaring the subject was lost to follow-up, three unsuccessful attempts at contact were made and recorded on the SAE form. The immediate and follow-up reports identified subjects by unique code numbers assigned to the trial subjects rather than by the subjects’ names, personal identification numbers, and/or addresses. The investigators were obligated to submit SAEs to the institutional review board or ethics committee according to their institutional review board or ethics committee guidelines (ICH-GCP E6). Drug-related SAEs would have been reported to the Food and Drug Administration by XBiotech’s Medical Safety Officer according to 21 CFR 312.32.

### Study Endpoints

The primary study endpoint was the clinical safety and tolerability of bermekimab in moderate-to- severe HS. The secondary endpoints were change in the Hospital Anxiety and Depression Scale and HiSCR, assessment of pharmacokinetics, change in patient-reported outcomes (VAS for disease, VAS for pain, and Dermatology Life Quality Index), assessment of PGA and Disease Activity Score, and change in inflammatory lesion (abscesses and inflammatory modules) count from baseline to week 12.

### Statistical Analysis

All analyses were summarized using descriptive statistics and data was presented in tabular format for each treatment group. Changes in outcomes for all endpoints from baseline to week 12 for both treatment groups were summarized using the number of observations available (n), mean, SD, median, range, and 95% confidence interval of the mean change from baseline. Categorical data was summarized for each treatment group using counts and percentages. Any missing patient data was imputed using Last Observation Carried Forward.

### Example 4

Abbreviations: HADS, Hospital Anxiety and Depression Scale; HiSCR, Hidradenitis suppurativa Clinical Response; HS, Hidradenitis suppurativa; PGA, Physician’s Global Assessment; SAE(s), serious adverse events; SC, Subcutaneous; DLQI, dermatology life quality index.

### Study to Characterize Efficacy of Bermekimab in Moderate to Severe Hidradenitis Suppurativa

The study is a Phase 2, randomized, double-blind, placebo-controlled study of bermekimab in patients with moderate to severe Hidradenitis Suppurativa. Participants are randomized in a 1:1:1 ratio to one of 3 arms: (i) 800 mg bermekimab administered subcutaneously at Weeks 0 and 1, followed by weekly subcutaneous administration of 400 mg bermekimab from Weeks 2 through 15 (treatment arm 1); (ii) 800 mg bermekimab administered subcutaneously at Weeks 0 and 1, followed by subcutaneous administration of 400 mg bermekimab once every two weeks from Weeks 2 through 15 (treatment arm 2); or (iii) placebo. Participants enter a 16-week extension and receive 400 mg bermekimab administered subcutaneously for 16 weeks.

### Study Endpoints

The primary endpoint of the study is the proportion of participants achieving HiSCR at Week 12. Participants are evaluated for efficacy using other measures including Pruritus Numerical Rating Scale, Pain Numerical Rating Scale, Hidradenitis Suppurativa Symptom Diary, HADS, DLQI, and HS-Physician’s Global Assessment.

### Subject Information and Baseline Characteristics

The majority of subjects were white (64.7%), while 28.1% black or African American, and 73.9% of the subjects were female. The mean age was 40, ranging 18 to 77 years. The average duration of HS was 9.1 years. The mean AN count was 11.7 with mean abscess and inflammatory nodule counts of 2.4 and 9.3 respectively. In addition, 60.8% of subjects presented with Hurley stage II disease at baseline. Overall, these baseline patient and disease characteristics were consistent with adalimumab phase 3 HS clinical trials. Baseline characteristics for enrolled patients are shown in the table below.

Summary of Demographic, HS Characteristics, and Patient Report Outcomes (DLQI, HSSD and Numerical rating Scale Diary) at Baseline

| | Placebo | Bermekimab | | | Total |
|---|---|---|---|---|---|
| | | 400 mg q2w | 400 mg qw | Combined | |
| Analysis set: Full analysis set | 52 | 50 | 51 | 101 | 153 |
| Weight Mean (SD) | 94.57 (24.064) | 94.08 (21.224) | 94.57 (24.064) | 94.33 (22.590) | 95.65 (23.652) |
| HS Characteristics | | | | | |
| Hurley stage | | | | | |
| II | 28 (53.8%) | 30 (60.0%) | 35 (68.6%) | 65 (64.4%) | 93 (60.8%) |
| III | 24 (46.2%) | 20 (40.0%) | 16 (31.4%) | 36 (35.6%) | 60 (39.2%) |
| HS disease duration (years) Mean (SD) | 8.4 (7.05) | 10.2 (10.49) | 8.8 (8.98) | 9.5 (9.73) | 9.1 (8.90) |
| AN Count Mean (SD) | 11.5 (9.07) | 13.4 (11.77) | 10.1 (7.49) | 11.8 (9.94) | 11.7 (9.62) |
| AN count [n(%)] | | | | | |
| <5 | 7 (13.5%) | 9 (18.0%) | 10 (19.6%) | 19 (18.8%) | 26 (17.0%) |
| 5-10 | 23 (44.2%) | 18 (36.0%) | 24 (47.1%) | 42 (41.6%) | 65 (42.5%) |
| ≥11 | 22 (42.3%) | 23 (46.0%) | 17 (33.3%) | 40 (39.6%) | 62 (40.5%) |
| Abscess count Mean (SD) | 2.0 (3.90) | 3.5 (7.49) | 1.7 (3.30) | 2.6 (5.81) | 2.4 (5.23) |
| Inflammatory nodule count Mean (SD) | 9.4 (7.80) | 9.9 (9.07) | 8.4 (5.62) | 9.2 (7.53) | 9.3 (7.60) |
| Draining fistula count Mean (SD) | 1.4 (2.49) | 2.7 (4.47) | 1.4 (3.05) | 2.1 (3.85) | 1.8 (3.46) |
| HIS4 Mean (SD) | 23.5 (19.33) | 32.1 (34.66) | 20.9 (20.11) | 26.5 (28.68) | 25.5 (25.85) |
| Baseline Patient Report Outcome | | | | | |
| DLQI Mean (SD) (0-30) | 15.7 (8.06) | 14.4 (7.26) | 13.0 (7.21) | 13.7 (7.23) | 14.4 (7.56) |
| HSSD symptom score Mean (SD) (0-10) | 6.1 (2.36) | 5.8 (2.10) | 5.6 (2.28) | 5.7 (2.18) | 5.8 (2.25) |
| HSSD HS related (past 24 hours) pain scale score (Mean (SD) (0-10) | 6.5 (2.80) | 6.3 (2.74) | 5.8 (2.99) | 6.1 (2.86) | 6.2 (2.84) |
| Weekly average of worst daily skin pain | | | | | |
| N | 42 | 43 | 41 | 84 | 126 |
| Mean (SD) | 6.30 (2.352) | 6.38 (2.142) | 6.31 (2.733) | 6.34 (2.434) | 6.33 (2.397) |
| ≥ 3 | 37 (88.1%) | 40 (93.0%) | 38 (92.7%) | 78 (92.9%) | 115 (91.3%) |
| Weekly average of average daily skin pain | | | | | |
| N | 42 | 43 | 41 | 84 | 126 |
| Mean (SD) | 5.52 (2.603) | 5.82 (2.299) | 5.32 (2.619) | 5.58 (2.458) | 5.56 (2.497) |
| ≥ 3 | 35 (85.4%) | 37 (92.5%) | 34 (77.3%) | 71 (84.5%) | 106 (84.8%) |
| Weekly average of worst daily skin itch | | | | | |
| N | 42 | 43 | 41 | 84 | 126 |
| Mean (SD) | 5.51 (2.939) | 5.40 (2.816) | 5.65 (3.095) | 5.52 (2.940) | 5.52 (2.928) |
| ≥ 3 | 35 (83.3%) | 32 (74.4%) | 33 (80.5%) | 65 (77.4%) | 100 (79.4%) |
| ≥ 4 | 31 (73.8%) | 29 (67.4%) | 28 (68.3%) | 57 (67.9%) | 88 (69.8%) |
| Weekly average of average daily skin itch | | | | | |
| N | 42 | 43 | 41 | 84 | 126 |
| Mean (SD) | 5.06 (2.750) | 4.71 (2.871) | 4.80 (2.898) | 4.76 (2.867) | 4.86 (2.821) |
| ≥ 3 | 34 (81.0%) | 29 (67.4%) | 31 (75.6%) | 60 (71.4%) | 94 (74.6%) |
| ≥ 4 | 29 (69.0%) | 25 (58.1%) | 24 (58.5%) | 49 (58.3%) | 78 (61.9%) |

In total, 13.7% (21/153) of subjects discontinued study up to Week 16. The proportion of subjects discontinuing study was 7.7% (4/52) in the placebo group, 18% (9/50) in the bermekimab 400 mg q2w group and 15.7% (8/51) in the bermekimab 400 mg qw group. The most common reason for discontinuation for all three groups were subjects withdrew consent (15.8% [3/52] in placebo group, and 10.0% [5/50], and 9.8% [5/51] in bermekimab 400 mg q2w and qw groups respectively).

In addition, among these 21 subjects who discontinued from the study, 6 subjects discontinued the study for the reason of “other” (1 in placebo group, 4 in bermekimab 400 mg q2w group, and 1 in bermekimab qw group ); and among these subjects, two (all in the bermekimab 400 mg q2w group) of the study discontinuations were COVID-19 related .

### Efficacy Analysis

### Primary Efficacy Endpoints

The proportion of participants achieving Hidradenitis Suppurativa Clinical Response (HiSCR) at Week 12:

For the primary endpoint analysis, subjects who discontinued treatment prior to Week 12 were considered HiSCR non-responders at Week 12. In addition, subjects with missing lesion count evaluation at Week 12 were considered as HiSCR non-responders at Week 12.

At Week 12, the proportion of subjects achieving a HiSCR in either bermekimab treatment group is not significantly different from that of the placebo group (see table below), although the proportion of subjects who achieved a HiSCR was numerically higher in the bermekimab 400 mg qw group (54.9%, p=0.339) than placebo (44.2%).

Proportion of Subjects Achieving HiSCR at Week 12; Full Analysis Set

| | Placebo | Bermekimab | | |
|---|---|---|---|---|
| | | 400 mg q2w | 400 mg qw | Combined |
| Analysis set: Full analysis set | 52 | 50 | 51 | 101 |
| Subjects achieving HiSCRad | 23 (44.2%) | 22 (44.0%) | 28 (54.9%) | 50 (49.5%) |
| Treatment difference (95% CI)^{b} | | -0.3% | 9.3% | 4.9% |
| | | (-18.5%, | (-8.4%, | (-10.6%, |
| | | 17.8%) | 26.9%) | 20.3%) |
| p-value^{c} | | 0.974 | 0.339 | |

^{a} Subjects with missing data or discontinued study treatment are assumed to be non-responders.
^{b} Treatment difference and 95% CI were calculated adjusting for prior biologic use (yes, no) and screening Hurley stage (II, III) using MH weights.
^{c} The p-values are based on CMH chi-square test stratified by prior biologic use (yes, no) and screening Hurley stage (II, III).
^{d} HiSCR is defined as at least 50% reduction in total abscess and inflammatory nodule count (AN count) with no increase in abscess count and no increase in draining fistula count relative to baseline.

### Supplementary Analyses

To assess the robustness of the results for the primary endpoint analysis, two supplementary analyses as specified below were conducted and the results of the supplementary analyses are presented:

Supplementary Analysis 1: Data after treatment discontinuation were considered as missing, missing data were then addressed by Multiple Imputation (shown in the table below).

Proportion of Subjects Achieving HiSCR at Week 12 (Supplementary Analysis 1); Full Analysis Set

| | Placebo | Bermekimab | | |
|---|---|---|---|---|
| | | 400 mg q2w | 400 mg qw | Combined |
| Analysis set: Full analysis set | 52 | 50 | 51 | 101 |
| Subjects achieving HiSCR^{ad} | 51.9% | 53.0% | 63.6% | 58.4% |
| Treatment difference (95% CI)^{b} | | 1.1% | 10.0% | 6.0% |
| | | (-20.1%, 22.4%) | (-10.1%, 30.1%) | (-11.9%, 23.9%) |
| p-value^{c} | | 0.916 | 0.330 | |

^{a} After subjects discontinued study treatment, the data was considered as missing. Multiple imputation method was used to impute missing data.
^{b} Treatment difference and 95% CI were calculated adjusting for prior biologic use (yes, no) and screening Hurley stage (II, III) using MH weights.
^{c} The p-values are based on CMH chi-square test stratified by prior biologic use (yes, no) and screening Hurley stage (II, III).
^{d} HiSCR is defined as at least 50% reduction in total abscess and inflammatory nodule count (AN count) with no increase in abscess count and no increase in draining fistula count relative to baseline.

Supplementary Analysis 2: Observed data at Week 12 were used regardless of whether the subject discontinued treatment or have missing data.

Across all treatment groups, the HiSCR response rates obtained from these two supplementary analyses were higher than the response rates shown in the primary analysis; but the treatment differences between the bermekimab groups and placebo group are similar across the primary analysis and the two supplementary analyses.

### Subgroup Analysis

To evaluate the consistency of the primary efficacy endpoint (proportion of participants who achieved HiSCR at Week 12) across demographics and baseline disease characteristics, subgroup analyses were also performed.

In general, similar to the primary analysis, when sample size permitted (≥10 in both treatment groups), numerically high HiSCR response rates were observed in bermekimab 400 mg qw group than placebo group across almost all demographic characteristics (sex, baseline age, baseline weight, race, and BMI) subgroups and baseline disease characteristics (baseline Hurley stage status, age at diagnosis, HS disease duration, baseline AN count, and prior biologic therapy used) while no clear difference was observed between bermekimab q2w and placebo.

### Secondary Efficacy Endpoints

Secondary endpoints are not controlled for multiplicity and the results of selected secondary endpoints are presented below. For binary endpoints, subjects who discontinued treatment for any reason were considered as non-responders from that point onward. The remaining missing data were also imputed as non-response; and for continuous endpoints, if a subject discontinued treatment, zero was assigned to improvement or percent improvement.

The proportion of subjects achieving HiSCR at Week 16:
At Week 16, numerically greater proportion of treated subjects randomized to bermekimab 400 mg q2w (52.0%, P=0.394) and 400 mg qw (56.9%, p=0.207) groups achieved a HiSCR compared with placebo (44.2%).

Change from baseline in HS-related pain in the past 24 hours based on Hidradenitis Suppurativa Symptom Diary (HSSD), AN Count, and DLQI at Week 12 and Week 16:
In general, no clear difference was observed between the bermekimab groups and the placebo group in the change from baseline in HS-related pain in the past 24 hours based on HSSD at Week 12 or Week 16.

Slightly greater reduction from baseline in AN count as measured by LSMeans was observed in the bermekimab groups than in the placebo group at Week 12 but this was not observed at Week 16.

Slightly greater reduction from baseline in DLQI as measured in LSMeans was observed in bermekimab 400 mg qw or q2w groups than placebo group at Week 12 and Week 16.

These results are shown in the table below:

Summary of Change from Baseline in HS-related pain in the past 24 hours, AN Count, Dermatology Life Quality Index (DLQI), and at Week 12 and Week 16; Full Analysis Set

| | Placebo | Bermekimab | |
|---|---|---|---|
| | | 400 mg q2w | 400 mg qw |
| Analysis set: Full analysis set | 52 | 50 | 51 |
| HSSD pain in past 24 hours (0-10) | | | |
| Baseline | | | |
| N | 52 | 50 | 51 |
| Mean (SD) | 6.5 (2.80) | 6.3 (2.74) | 5.8 (2.99) |
| Change from baseline^{a} Week 12 | | | |
| N | 51 | 48 | 50 |
| LSMean 95% CI)^{c} | -1.8 (-2.57, -1.02) | -1.6 (-2.39, -0.82) | -1.9 (-2.67, -1.12) |
| LSMean difference (95% CI)^{c} | | 0.2 (-0.78, 1.15) | -0.1 (-1.07, 0.86) |
| p-value^{c} | | 0.700 | 0.827 |
| Week 16 | | | |
| N | 51 | 50 | 50 |
| LSMean (95% CI)^{c} | -1.8 (-2.65, -1.01) | -1.7 (-2.54, -0.90) | -1.8 (-2.66, -1.02) |
| LSMean difference (95% CI)^{c} | | 0.1 (-0.90, 1.12) | -0.0 (-1.02, 1.01) |
| p-value^{c} | | 0.830 | 0.989 |
| AN count Baseline | | | |
| N | 52 | 50 | 51 |
| Mean (SD) | 11.5 (9.07) | 13.4 (11.77) | 10.1 (7.49) |
| Change from baseline^{ab} Week 12 | | | |
| N | 48 | 45 | 47 |
| LSMean (95% CI)^{c} | -2.6 (-4.77, -0.43) | -3.2 (-5.48, -0.94) | -4.3 (-6.46, -2.08) |
| LSMean difference (95% CI)^{c} | | -0.6 (-3.30, 2.08) | -1.7 (-4.34, 1.00) |
| p-value^{c} | | 0.655 | 0.217 |
| Week 16 | | | |
| N | 48 | 46 | 47 |
| LSMean (95% CI)^{C} | -4.5 -6.65, -2.45 | -3.0 -5.23, -0.83 | -4.7 (-6.81, -2.60) |
| LSMean difference (95% CI)^{c} | | 1.5 (-1.09, 4.13) | -0.2 (-2.75, 2.43) |
| p-value^{c} | | 0.253 | 0.904 |
| DLQI (0-30) Baseline | | | |
| N | 52 | 50 | 51 |
| Mean | 15.7 (8.06) | 14.4 (7.26) | 13.0 (7.21) |
| Change from baseline^{a} Week 12 | | | |
| N | 50 | 48 | 49 |
| LSMean (95% CI)^{c} | -2.3 (-3.92, -0.65) | -3.4 (-5.05, -1.78) | -4.0 (-5.58, -2.33) |
| LSMean difference (95% CI)^{c} | | -1.1 (-3.14, 0.89) | -1.7 (-3.69, 0.37) |
| p-value^{c} | | 0.272 | 0.108 |
| Week 16 | | | |
| N | 52 | 50 | 50 |
| LSMean (95% CI)^{c} | -2.7 (-4.44, -1.02) | -4.2 (-5.88, -2.46) | -4.1 (-5.83, -2.41) |
| LSMean difference (95% CI)^{c} | | -1.4 (-3.55, 0.67) | -1.4 (-3.52, 0.73) |
| p-value^{c} | | 0.180 | 0.197 |

^{a} The analysis is based on observed data or 0 (no improvement) if a subject discontinued study treatment.
^{b} AN count is the total of abscess and inflammatory nodule counts.
^{c} LSMeans, LSMean differences, and p-values are based on the MMRM model with treatment, visit, the interaction of treatment group and visit, baseline values, prior biologic use (yes, no), screening Hurley stage (II, III), prior biologic use by visit, Hurley stage by visit, and the interaction of baseline value and visit as covariates.

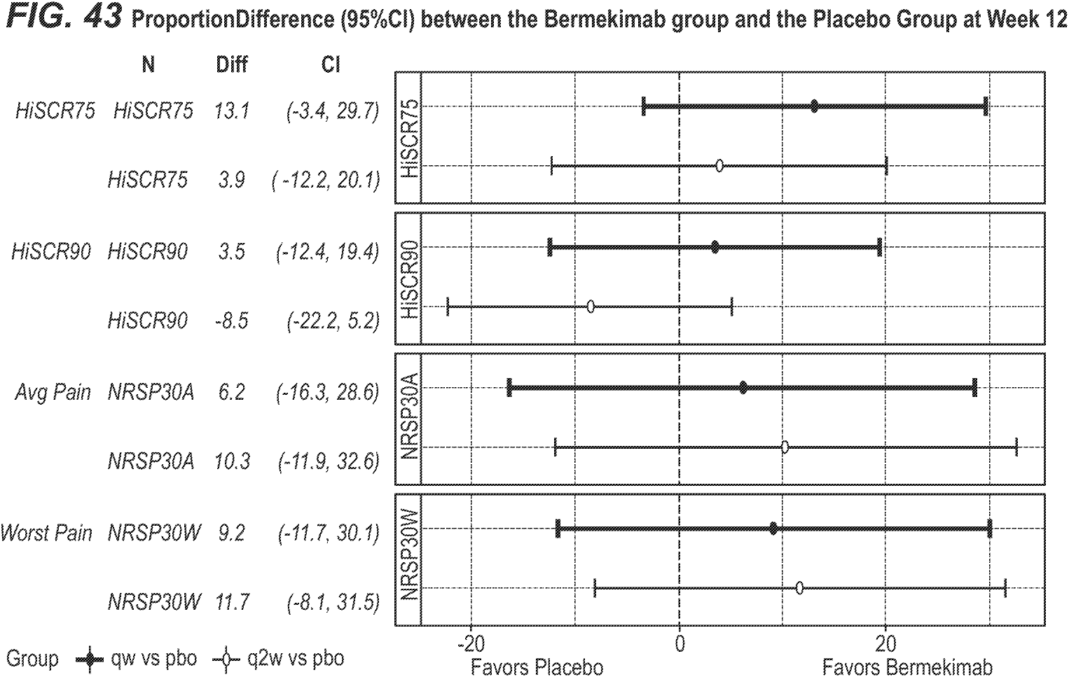

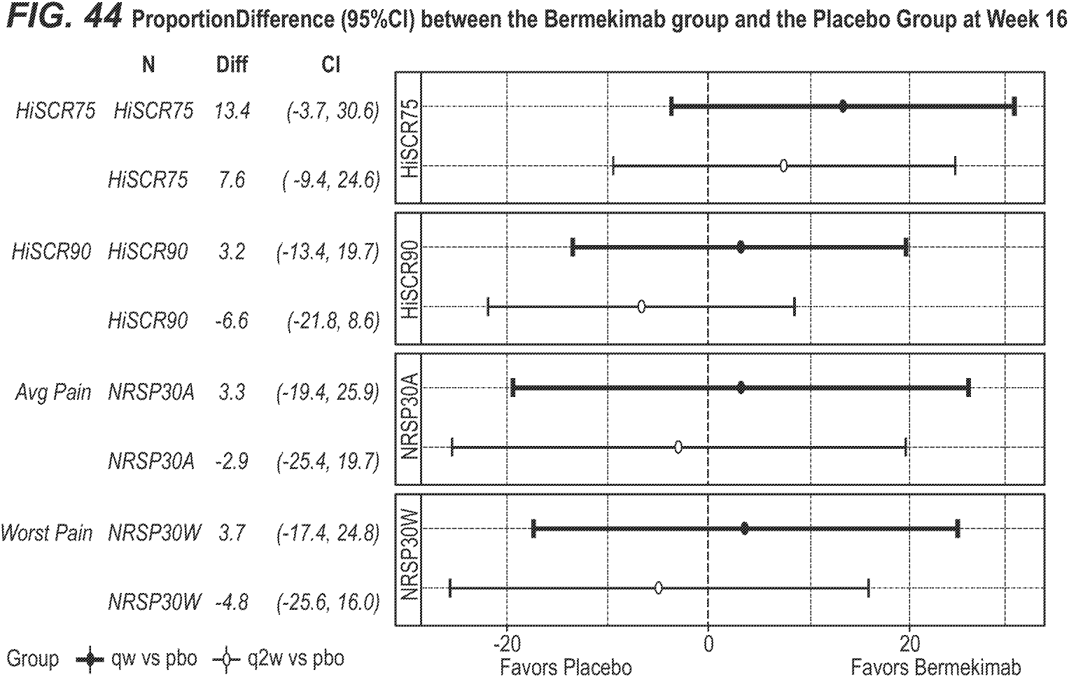

Proportion of Subjects Achieving HiSCR75 response, HiSCR90 response, NRS30 in NRS Weekly Average of daily worst and daily Average Skin Pain Scores:
HiSCR75 and HiSCR90 is defined as at least 75% and 90% reduction in total abscess and inflammatory nodule count (AN count) respectively with no increase in abscess count and no increase in draining fistula count relative to baseline
NRS30 is defined as at least 30% reduction from baseline in numerical rating scale for pain among subjects with NRS skin pain scores ≥3at baseline
Numerically higher response rates were observed in the bermekimab qw regimen as compared with placebo across all 4 endpoints and at both timepoints though all of the 95% CIs on the proportion difference contain 0. No consistent results were observed comparing the bermekimab q2w regimen with the placebo group. Forest plots with treatment differences and the corresponding 95% CIs between the bermekimab groups and the placebo group at Week 12 and Week 16 are presented in FIG. 43 and FIG. 44 respectively.

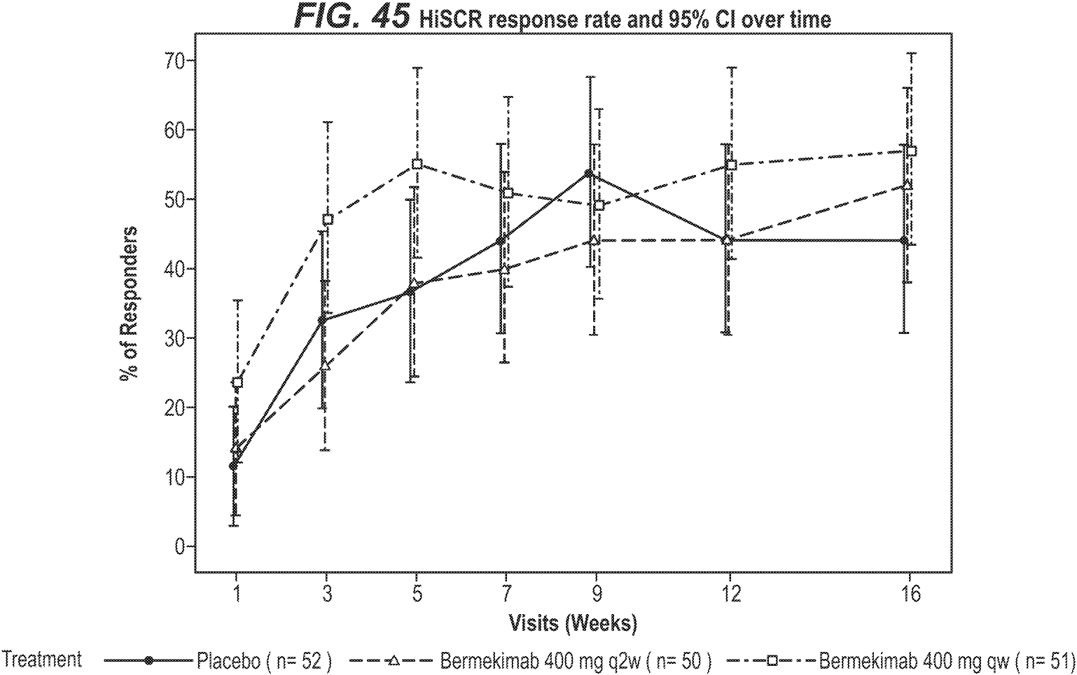

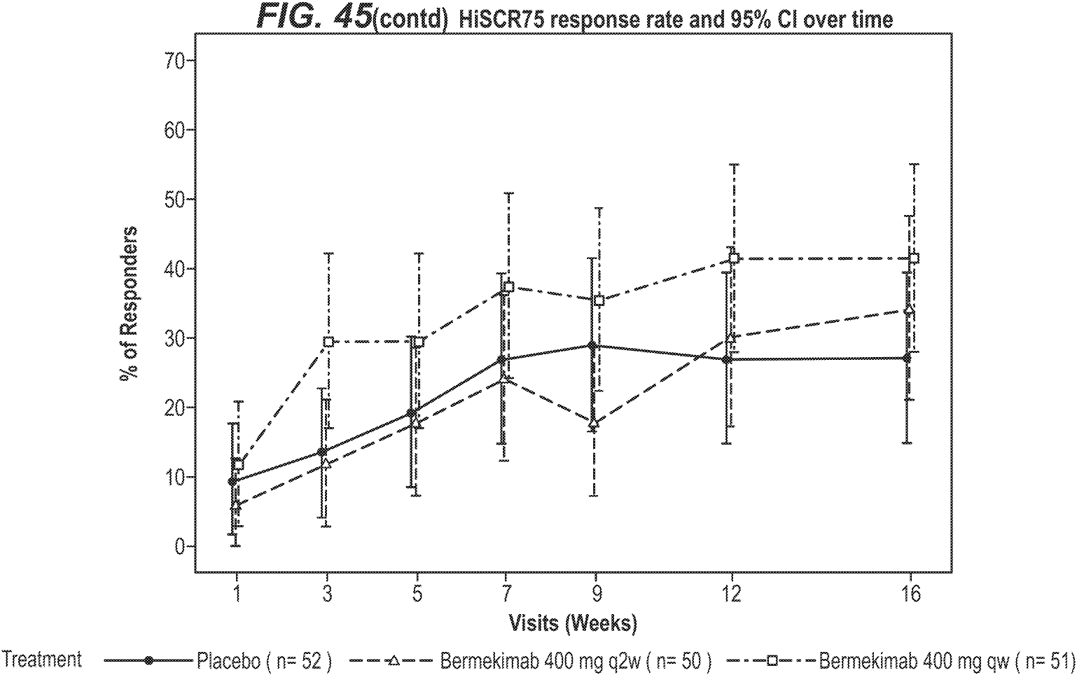

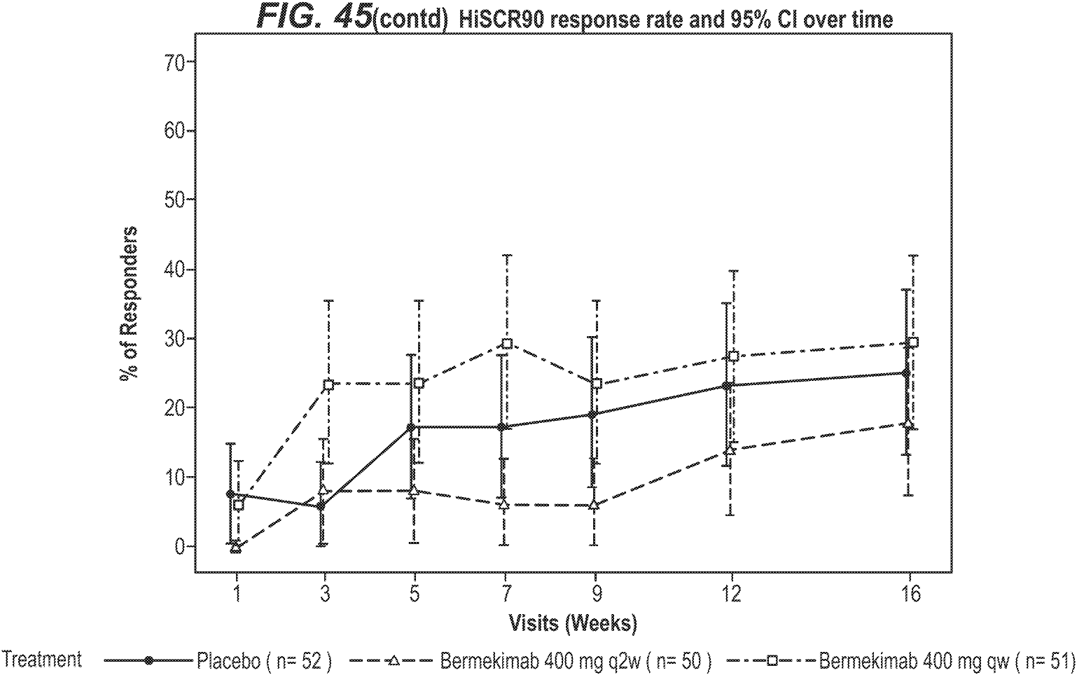

HiSCR, HiSCR75, andHiSCR90 over time through week 16:
The proportions and the corresponding 95% CIs of subjects achieving HiSCR, HiSCR75, and HiSCR90 over time from Week 1 through Week 16 are summarized in FIG. 45.

Between Weeks 1 and 16, HiSCR response rates of bermekimab 400 mg qw group are slightly higher than both the bermekimab 400 mg q2w and placebo groups. No clear pattern of difference over time was observed between bermekimab 400 mg q2w and placebo groups.

Similar response pattern was observed for HiSCR75 and HiSCR90; except that lower Hi SCR90 response rates were observed in bermekimab 400 mg q2w group than placebo group over time through Week 16.

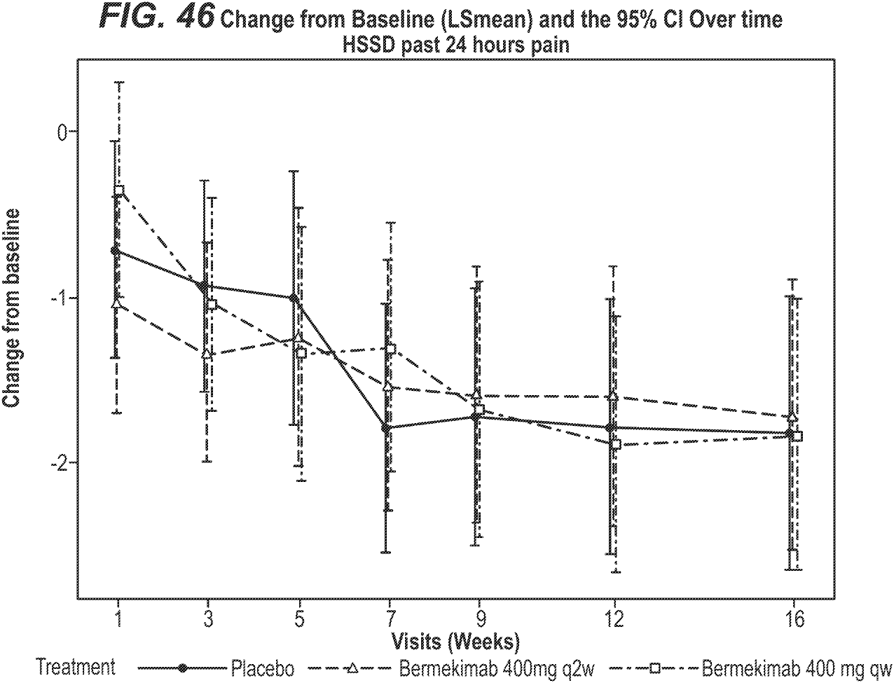

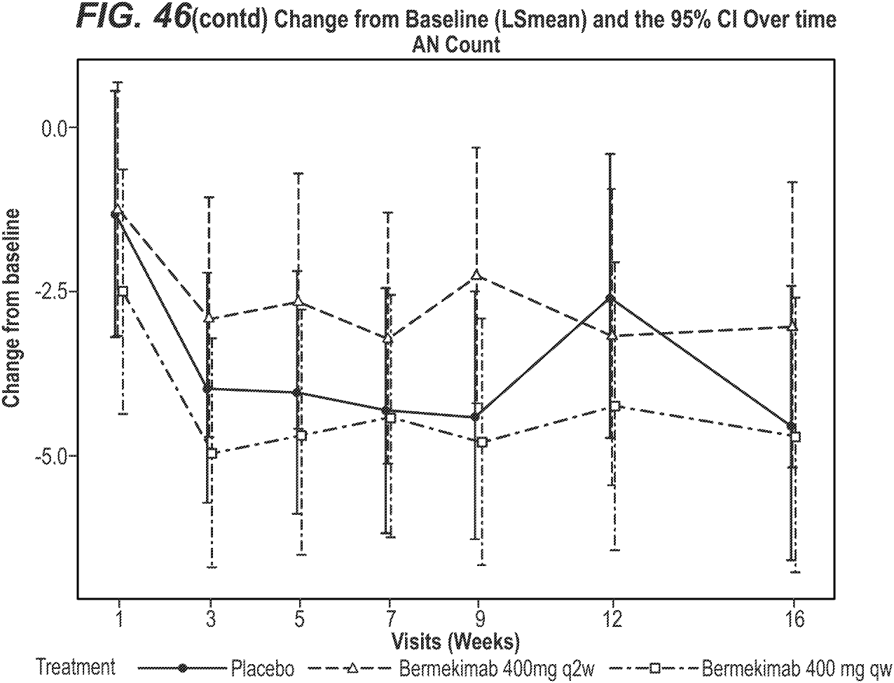

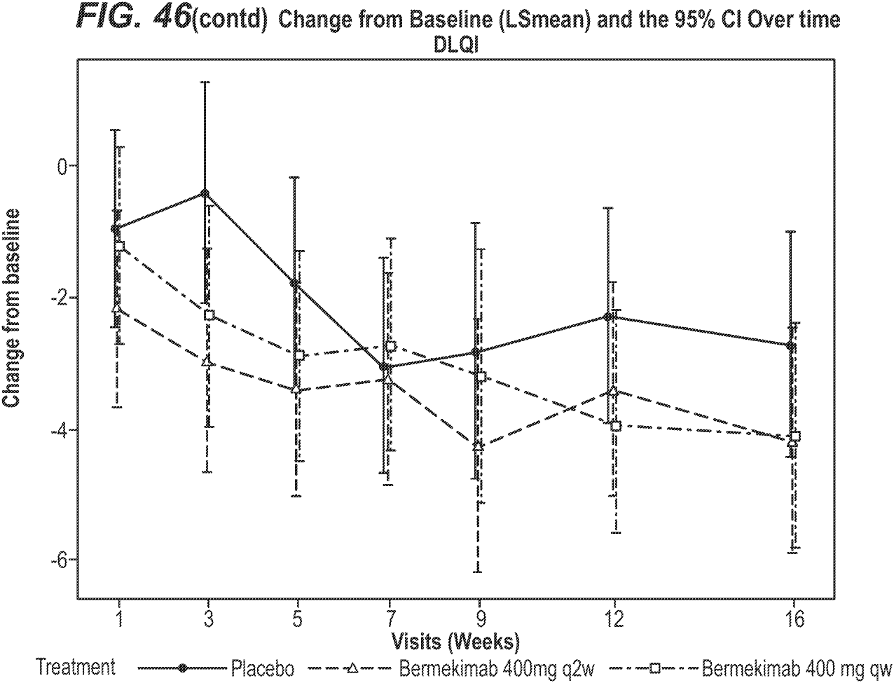

Change from baseline in HS-related skin pain in the Past 24 hours, AN count, and DLQI over time through week 16:
LSMeans and the corresponding 95% CIs for change from baseline in HSSD past 24 hours pain, AN count and DLQI were presented in FIG. 46.

No clear pattern of difference over time was observed between the bermekimab groups and the placebo group in the change from baseline in HSSD skin pain in the past 24 hours. Slightly greater reduction from baseline was observed in AN count in the bermekimab qw regimen when compared with the placebo group over time through Week 16 while the reduction from baseline in AN count appeared to be smaller in the bermekimab q2w group than the placebo group over time. Slightly greater reduction from baseline in DLQI was generally observed in both bermekimab groups when compared with the placebo group over time through Week 16.

### Summary of Results

The proportion of subjects achieving a HiSCR50 response at Week 12 was numerically greater in the bermekimab 400 mg qw (54.9%, p=0.339) group relative to placebo (44.2%), while no difference was observed for the 400 mg q2w (44.0%, p=0.974) group.

At Week 16, numerically greater proportions of subjects randomized to the 400 mg qw (56.9%, p=0.207) and 400 mg q2w (52.0%, p=0.394) groups achieved a HiSCR response relative to placebo (44.2%).

Placebo rates were high compared with prior HS studies for the primary endpoint of HiSCR50 (44%) as well as more stringent endpoints of HiSCR75 (26.9%) and HiSCR90 (23 \. 1%) that may have obscured the ability to discriminate drug effect of bermekimab in HS. In comparison, recent bimekizumab trials had placebo rates of HiSCR50 (23.1%), HiSCR75 (11%) and HiSCR90 (0%).

No clear difference was observed between the bermekimab groups and the placebo group in the change from baseline in HS-related pain in the past 24 hours at Week 12.

Numerically greater reductions from baseline in DLQI score were observed in the bermekimab 400 mg qw and 400 mg q2w groups compared to the placebo group at Week 12 which did not reach statistical significance.

An apparent exposure-response relationship was observed for HiSCR50 at Weeks 5, 12 and 16 in bermekimab 400 mg QW dose group while not in 400 mg Q2W group; albeit the number of subjects in each serum concentration quartile was small.

The inter-subject variability in observed serum drug concentrations was relatively high (50-100%), albeit not unusual for SC mAbs.

In addition to relatively higher variability in the PK data, anomalous individual drug concentration values were observed that suggest potential medication administration errors and/or errors in the sample handling at several sites: 2 of 49 placebo subjects at different sites demonstrated bermekimab exposure; 2 of 29 bermekimab qw subjects at a single site had no detectable bermekimab upon repeated and confirmed sample analysis.

### Safety

The adverse events (AEs) observed in the HS trial are consistent with published studies, and no safety concerns were observed. The most common AEs were nasopharyngitis 5.0% (5 subjects) in the combined bermekimab group and 3.8% (2 subjects) in the placebo group; and upper respiratory tract infection 4.0% (4 subjects) in the combined bermekimab group and 1.9% (1 subject) in the placebo group. Injection site erythema was observed in 5% (5 subjects) of subjects in the combined bermekimab group and no subjects in the placebo group.

AEs in the System Organ Class (SOC) of infections and infestations were most commonly reported, with 12 subjects (23 \. 1%) reporting AEs in the placebo group, 13 subjects (25.5%) in bermekimab 400 mg qw group and 10 subjects (20.0%) in bermekimab 400 mg q2w group. There were no dose related infection adverse events observed.

SAEs were reported in 1 (1.0%) subject (injection site erythema and swelling) in the combined bermekimab group and 2 (3.8%) subjects in the placebo group (sinus tachycardia and epilepsy).

Safety was assessed among all randomized and treated subjects who received at least 1 dose of study agent (partial or complete) according to the assigned treatment received during the study, irrespective of the treatment assigned at randomization. This is also referred to as the safety analysis set. Key safety events are summarized in the table below.

Key safety events through Week 16
Bermekimab

| | Placebo | 400 mg q2w | 400 mg qw | Combined |
|---|---|---|---|---|
| Analysis set: Safety analysis set | 52 | 50 | 51 | 101 |
| Avg duration of follow-up (weeks) | 15.3 | 14.7 | 14.7 | 14.7 |
| Avg exposure (number of administrations) | 14.7 | 14.3 | 14.4 | 14.4 |
| Subjects who discontinued study agent because of 1 or more adverse events | 1 (1.9%) | 2 (4.0%) | 2 (3.9%) | 4 (4.0%) |
| Subjects with 1 or more: | | | | |
| Adverse events | 25 (48.1%) | 27 (54.0%) | 24 (47.1%) | 51 (50.5%) |
| Serious adverse events | 2 (3.8%) | 0 | 1 (2.0%) | 1 (1.0%) |
| Death | 0 | 0 | 0 | 0 |

The proportion of subjects experiencing 1 or more AEs was 50.5% in the combined bermekimab group and 48.1% in the placebo group. The most frequent system organ-class involved in AEs in all groups was infections and infestations (22.8% in the combined bermekimab group and 23.1% in the placebo group. The most common AEs in this SOC were nasopharyngitis (5.0% (n=5) in the combined bermekimab group and 3.8% (n=2) in the placebo group), and upper respiratory tract infection (4.0%) in the combined bermekimab group and 1.9% in the placebo group). Notably difference of AE injection site erythema was observed between combined bermekimab and placebo groups (5% vs 0% respectively). The percentage of subjects with one or more serious adverse events (SAEs) through Week 16 was 3.8% (n=2) in the placebo group, 1.0% (n=1) in the combined bermekimab group. No death occurred through week 16.

### Conclusions

These results suggest that bermekimab 400 mg qw may have modest efficacy in patients with hidradenitis suppurativa, though the treatment effect did not reach statistical significance.

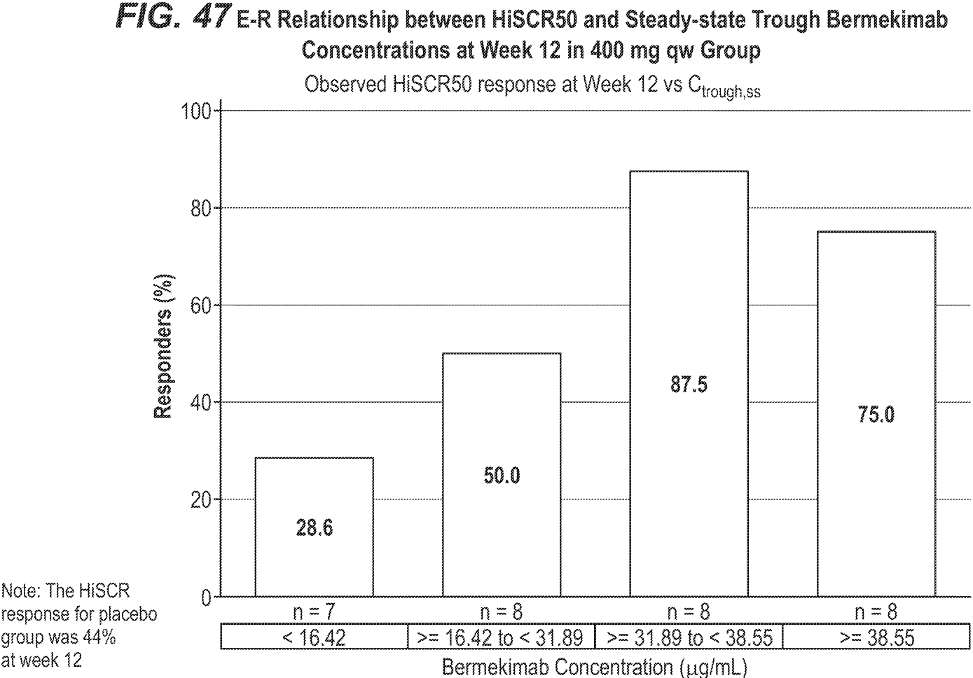

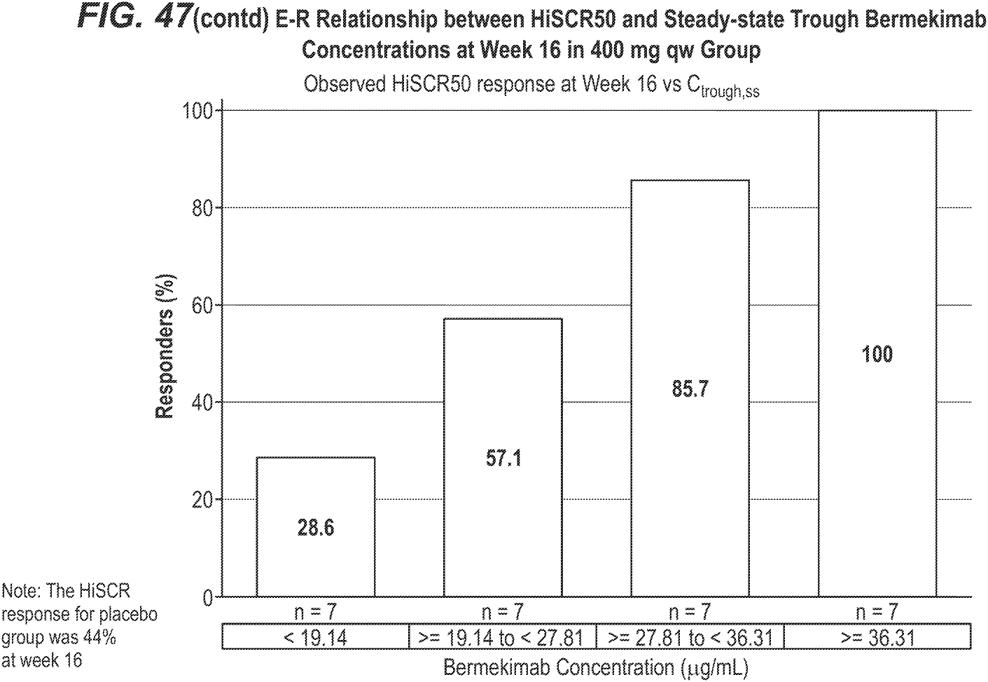

Exposure-response analysis indicated higher response rates in HiSCR50 with higher bermekimab plasma concentrations (FIG. 47), suggesting higher response is achievable at higher dose levels.

Example 5 - A Phase 1 Study to Investigate the Pharmacokinetics and Pharmacodynamics of bermekimab in Healthy Participants

This is an open-label, interventional study in healthy participants. Single doses of bermekimab are administered. Three doses of anakinra, the positive PD control, are administered.

There are single-dose SC cohorts and 3 single-dose IV cohorts. Participants are enrolled into either the SC cohorts (400 mg dose at concentrations of 100, 150, 175, and 200 mg/mL; 200 and 800 mg dose at concentration 175 mg/mL) or the IV cohorts (400, 800, and 1,200 mg at 100 mg/mL). There is also a cohort of that receives a 100 mg SC dose of anakinra daily for 3 days to be used as a PD comparator.

The total duration of participation is approximately 16 weeks, including a screening visit up to 28 days prior to study intervention administration. Participants have an inpatient period consisting of 9 days/8 nights. Participants return to the study site at Weeks 2, 3, 4, 6, 8, and 12.

Description of Interventions

| | | |
|---|---|---|
| Intervention Name | bermekimab | Anakinra |
| Dose Formulation | Liquid formulation; bermekiman; Trehalose Dihydrate; Sodium Phosphate Dibasic; Citric Acid Monohydrate, Phosphoric Acid; Sodium Hydroxide, Water for Injection | Anakinra is supplied in single-use preservative free, prefilled glass syringes with 29-gauge needles. Each prefilled glass syringe contains 100 mg of anakinra per 0.67 mL. The full syringe contains 100 mg anakinra. Citric acid, anhydrous; Sodium chloride; Disodium edetate dihydrate; Polysorbate 80; Sodium hydroxide; Water for injections |
| Unit Dose Strength(s) | Subcutaneous (SC): 100 mg/mL, 150 mg/mL, 175 mg/mL, 200 mg/mL intravenous (IV): 100 mg/mL | Injection: 100 mg/0.67 mL solution in a single-use prefilled syringe for SC injection. Graduated syringe allows for doses between 20 and 100 mg. |
| Dosage Level(s) | SC: 400 mg at 100 mg/mL, 400 mg at 150 mg/mL, 400 mg at 175 mg/mL, 400 mg at 200 mg/mL, 200 mg at 175 mg/mL, 800 mg at 175 mg/mL IV: 400, 800, 1,200 mg | 100 mg |
| Route of Administration | SC injection and IV infusion | SC injection |

Objectives and Endpoints

| Objectives | Assessments |
|---|---|
| Primary | |
| To assess the pharmacokinetics (PK) of bermekimab after single subcutaneous (SC) or intravenous (IV) administrations and the effect of formulation concentrations on PK of bermekimab in healthy participants. | PK parameters of bermekimab, including but not limited to Cₘₐₓ, AUC_{inf}, AUCₗₐₛₜ, T_{½} and F(%) |
| Secondary | |
| To evaluate safety, tolerability, and immunogenicity of a single SC or IV administration of bermekimab in healthy participants. | Proportion of participants with treatment-emergent adverse events (TEAE) by severity and serious adverse events (SAE) through Day 85. Clinically significant changes in vital signs, electrocardiograms (ECGs), hematology, chemistry, and urinalysis. Presence of antibodies to bermekimab. |
| Exploratory | |
| To assess pharmacodynamic (PD) modulation of interleukin-1 alpha (IL-1α) in comparison to a known antagonist of IL-1α and β by provoking the release of IL-1α in healthy skin and assessing the effect of blockade on downstream PD readouts. | Biopsy, incubation, and molecular endpoint assessment in skin biopsies and secreted cytokines before and after treatment with bermekimab or anakinra. |

This study employs a 2-wave dosing scheme. Wave 1 consists of Cohorts A through E and Wave 2 consists of Cohorts F through J. The cohorts in Wave 1 are randomized and dosed in a parallel manner according to site logistics. The cohorts in Wave 2 are not randomized but are enrolled in sequential order, ie, Cohort F is fully enrolled before starting Cohort G enrollment and so on.

| | |
|---|---|
| Wave 1 (Randomized Parallel Cohorts) | Wave 2 (Non-Randomized Sequential Cohorts) |
| Cohort A - 400 mg SC (formulation: 100 mg/mL) | Cohort F - 200 mg SC (formulation: 175 mg/mL) |
| Cohort B - 400 mg SC (formulation: 150 mg/mL) | Cohort G - 800 mg SC (formulation: 175 mg/mL) |
| Cohort C - 400 mg SC (formulation: 175 mg/mL) | Cohort H - 800 mg IV (formulation: 100 mg/mL) |
| Cohort D - 400 mg SC (formulation: 200 mg/mL) | Cohort I - 1,200 mg IV (formulation: 100 mg/mL) |
| Cohort E - 400 mg IV (formulation: 100 mg/mL) | Cohort J - 100 mg SC anakinra |

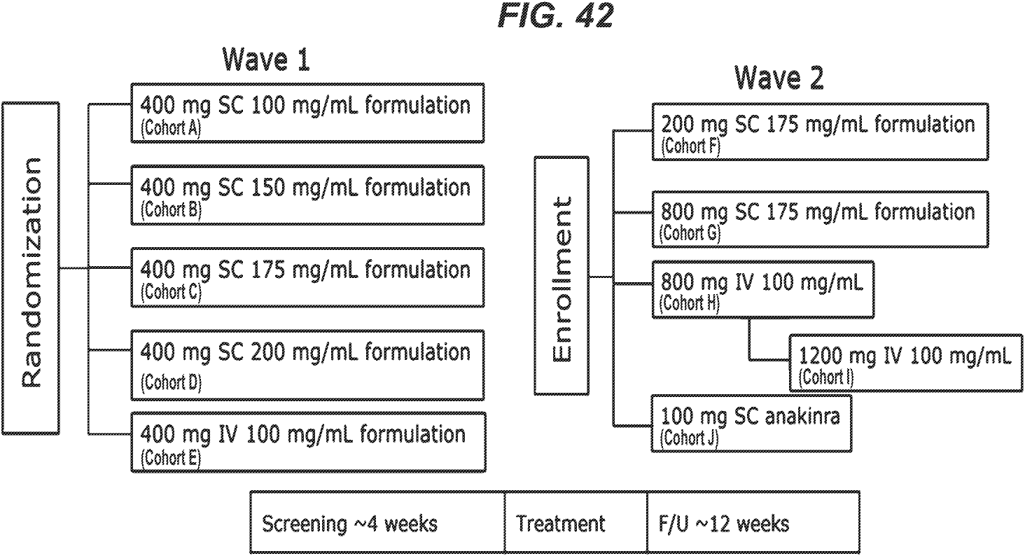

FIG. 42 is a schematic summarizing the design of the Phase 1 study.

### Pharmacokinetics

Serum and plasma samples are analyzed to determine concentrations of bermekimab using a validated, specific, and sensitive immunoassay method.

Pharmacokinetic parameters of bermekimab are calculated from concentrations over time data using noncompartmental analyses. Pharmacokinetic parameters following a single IV or SC administration of bermekimab include, but are not limited to:

IV only:
Cₘₐₓ: maximum observed plasma concentration.
AUC_{inf}: area under the plasma concentration versus time curve from time zero to infinity with extrapolation of the terminal phase.
AUCₗₐₛₜ: area under the plasma concentration versus time curve from time zero to the time corresponding to the last quantifiable concentration.
T_{½}: terminal half-life.
CL: total systemic clearance.
V_{z}: volume of distribution based on terminal phase.

SC only:
Cₘₐₓ: maximum observed plasma concentration.
Tₘₐₓ: time to reach maximum observed plasma concentration.
AUC_{inf}: area under the plasma concentration versus time curve from time zero to infinity with extrapolation of the terminal phase.
AUCₗₐₛₜ: area under the plasma concentration versus time curve from time zero to the time corresponding to the last quantifiable concentration.
T_{½}: terminal half-life.
CL/F: apparent total systemic clearance after extravascular administration.
V_{z}/F: apparent volume of distribution based on terminal phase after extravascular administration.
F(%): absolute SC bioavailability to be calculated using the following equation.

$$F \left(\%\right) = \frac{A U {C}_{i n f , S C}}{m e a n A U {C}_{i n f , I V}} \times 100 \%$$

### Pharmacodynamics

Participants are required to have 4, 4-mm skin punch biopsies of normal healthy skin (2 pre- and 2 posttreatment). Assessing the pattern of gene expression and protein production in the skin biopsies allows measurement of PD effect of bermekimab or anakinra (which serves as a positive control) on molecular events that have an established dependence on IL-1α. At each skin biopsy collection visit, one skin biopsy is processed immediately as per skin biopsy lab manual and the second skin biopsy is cultured ex vivo for 24 hours to establish the stimulation baseline for each participant (the stimulus is the skin biopsy procedure itself). The third and fourth skin biopsies are collected after bermekimab administration and 1 skin biopsy specimen is processed immediately as per skin biopsy laboratory manual and the second skin biopsy specimen is cultured ex vivo for 24 hours. The effects of bermekimab or anakinra dosing on gene and protein expression patterns induced as a result of the tissue injury from the skin biopsy collection procedure are determined by comparing changes relative to baseline in predefined gene expression signatures and secreted proteins in the supernatants. Skin biopsy specimens are assessed for gene expression and for secreted proteins accumulation in ex vivo culture supernatants.

Data from skin biopsy samples is analyzed to evaluate PD effects of each intervention on the induction of gene expression changes and secreted protein levels in response to the wounding process (biopsy procedure). Pharmacodynamic data is presented using suitable descriptive statistics for each intervention, comparing PD readouts in ex vivo-cultured pretreatment biopsies versus ex vivo-cultured posttreatment biopsies (biopsy samples that are not cultured ex vivo serve as control for changes occurring during ex vivo incubation). For each PD parameter (eg, expression levels of select genes, concentrations of protein readouts; % inhibition), summary plots (eg, mean and standard deviation or median and interquartile ranges) of absolute levels and changes from pretreatment are generated for each cohort.

Example 6 - Multicenter, randomized, double blind, placebo and active comparator-controlled dose ranging phase 2b study to evaluate the efficacy of bermekimab in participants with moderate to severe Hidradenitis Suppurativa (HS).

The study includes up to 5 treatment/dose arms, which include weekly (QW) subcutaneous administration (SC) of bermekimab (BMK; at three doses of 350, 700 and 1050 mg), adalimumab (ADA) and placebo. The active reference arm (adalimumab) is included to confirm validity of the trial, to serve as an internal reference for bermekimab clinical efficacy, and to validate the utility of exploratory assessments with a proven therapy.

The study includes 150 participants up to the interim analysis (IA), and a total of 290 participants as follows:
a. 70 subjects in placebo/bermekimab 1050 mg/adalimumab groups
b. 40/arm for bermekimab 350 mg and 700 mg group

The study is adequately powered to detect efficacy between active and placebo (pairwise comparison) and dose response using data across all treatment groups as well as the efficacy assessment of bermekimab vs adalimumab

One decisional IA is performed when approximately 150 (50/arm) subjects reach week 16 for placebo, bermekimab 1050 mg and adalimumab groups. The results of this IA could determine the subsequent action for the next cohort. Another preliminary IA is performed when 75 subjects (25/arm) reach Week 16 for placebo, bermekimab 1050 mg and adalimumab groups. Opening the subsequent cohort could be accelerated based on the results from the preliminary IA results. Interim analyses are based on clinical outcomes (i.e. HiSCR achieving TPP or better at the top dose)The study participants have moderate to severe HS and are enrolled based on the following disease related key inclusion criteria:
a. Subjects with moderate to severe HS for at least 1 year prior to the baseline visit as determined by the investigator through patient interview and/or review of the medical history.
b. HS lesions present in at least 2 distinct anatomic areas (examples include but are not limited to left and right axilla; or left axilla and left inguinocrural fold).
c. Inadequate response to an adequate course of appropriate oral antibiotics for treatment of HS (or demonstrated intolerance to, or had a contraindication to oral antibiotics for treatment of their HS) in the investigator’s opinion.
d. Stable HS for at least 1 month prior to the screening visit as determined by the investigator through patient interview and/or review of the medical history.
e. Total abscess and inflammatory nodule (AN) count of ≥5 at the screening and baseline visit.
f. Subject must agree to daily use (throughout the entirety of the study) of one of the following over-the-counter treatments to the body areas affected with HS lesions: either soap and water, or a topical antiseptic wash containing chlorhexidine gluconate, triclosan, or benzoyl peroxide, or a dilute bleach bath.

Key exclusion criteria - the subject has:
a. a draining fistula count of >20 at the baseline visit.
b. any other active skin disease or condition (e.g., bacterial, fungal, or viral infection) that could have interfered with assessment of HS.
c. received prescription topical therapies for the treatment if HS within 14 days prior to the baseline visit.
d. received systemic non-biologic therapies for the treatment of HS 28 days prior to the baseline visit.
e. received oral antibiotic treatment of HS or inflammatory disorders within 28 days prior to the baseline visit.
f. received opioid analgesics (including tramadol) within 14 days prior to the baseline visit, or if it is anticipated that the participant will require initiation of opioid analgesics (excluding tramadol) for any reason during the study period.
g. received PRN of “as needed” dose of oral concomitant analgesics for HS-related pain within 14 days prior to the baseline visit (participant may be receiving non-opioid analgesics for treatment of chronic non-HS-related or HS-related pain, but must be on a stable dose for at least 14 days prior to the baseline visit and be expected to continue use throughout the study).

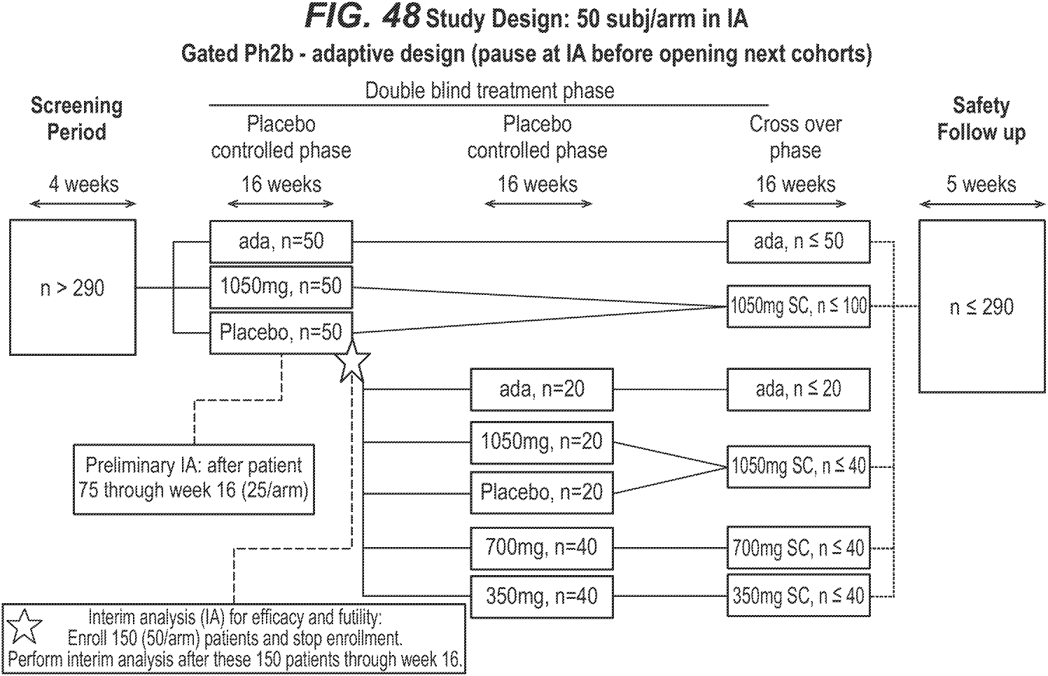

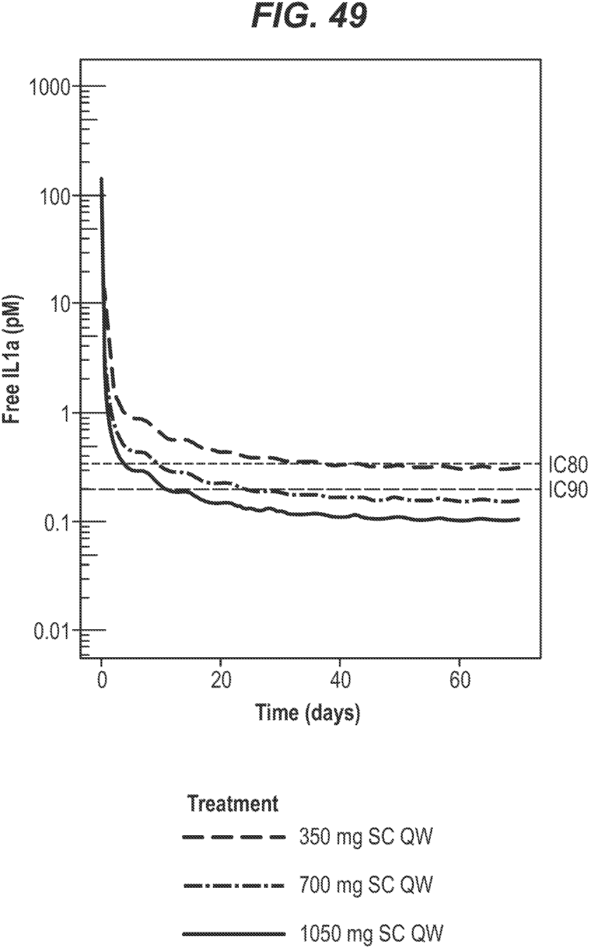

FIG. 48 is a schematic summarizing the design of the Phase 2 study.

### Study Endpoints

Primary endpoint: HiSCR50

Secondary endpoints used to assess the totality of Bermekimab efficacy in Ph2b:
a. Physician assessed measures: IHS4 (HS severity score), IGA (investigator Global Assessment)
b. Patient reported outcomes (PRO):
   i. DLQI: symptoms and feelings, daily activities, leisure, work or school performance, personal relationships, and treatment,
   ii. PROMIS-29: depression; anxiety; physical function; pain interference; fatigue; sleep disturbance; and ability to participate in social roles and activities
   iii. PGIS: disease severity
   iv. PGIC: disease improvement

Exploratory endpoints:
a. HSSD (pain, tenderness, swelling, heat, pressure, itch, odor, drainage and flu-like symptoms)
b. HASI: (Hidradenitis Suppurativa Area and Severity Index)
c. Digital endpoints: (SWIFT and Spectrum IR, for objectively monitoring disease activity)

### Interim Analysis Characteristics

The phase 2b study employs an adaptive design, in which dose assignments depend on the results of the interim analysis. At the beginning of the study, 150 subjects (50 subjects/arm) are randomized to receive BMK (1050 mg), ADA or placebo group at 1:1:1 ratio. The enrolment stops when 150 subjects are randomized. An interim analysis takes place when these subjects reach week 16. If the futility criterion is met, the study is stopped and it could trigger other activities such as transnational medicine study. If the study results land in the ‘course correction’ territory, comprehensive evaluations and appropriate course correction activity takes place. If the success criterion is met, the study continues with the next cohort in which randomization to BMK 1050 mg, ADA, placebo, BMK 700 mg and BMK 350 mg group at 1:1:1:2:2 ratio begins. Thus, if the study continues, a total number of 290 subjects are randomized to BMK1050mg, 700 mg, 350 mg, ADA or placebo. Additionally, a preliminary interim analysis is performed when initial 75 subjects (25 subjects/arm) reached week 16 to determine whether the results are very promising to enable early opening of the next cohort to minimize the enrolment pause.

### Dose Selection Rationale

### Pharmacokinetic / Target Engagement (PK / TE) Modeling

A minimal physiologically-based PK (mPBPK) modeling of skin IL-1α inhibition was used to estimate theoretical free IL-1α in skin at 350 mg QW, 700 mg QW and 1050 mg QW.

The following key assumptions made for mPBPK model are based on data generated ex vivo from skin (including Atopic Dermatitis tissue) and theoretical/measured IL-1 pathway mediator concentrations in HS skin:
Baseline skin interstitial fluid (ISF) concentrations:
   IL-1α = 146 (median) pM
   IL1RA = 960 (median) pM
   sIL1-R1 = ⅒ of IL1RA = 96 pM (based on an assessment that sIL-1R1 << IL-1RA)
   Potential impact of levels of IL-1β and IL1R2 are not considered in this preliminary simulation
Elimination rates of IL-1α, IL-1RA, sIL-1R1 equal 2.5 times the lymph flow rate
The production of IL-1α in skin ISF assumed stationary, i.e. no change with treatment
Bermekimab K_{D} = 0.059 nM
In vitro skin cells functional assays suggest
   IC80 of IL-1α = 0.3 pM
   IC90 of IL-1α = 0.2 pM

Assuming a baseline skin IL-1α level of 146 pM, 700 mg QW and 1050 mg QW are predicted to suppress free IL-1α in skin to below IC80 (0.3 pM), while 350 mg QW is predicted to be above 0.3 pM. Assuming a baseline skin IL-1α level of 146 pM, 700 mg and 1050 mg QW are predicted to suppress free IL-1α in skin to near IC90 (0.2 pM). The predictions from this model are shown in FIG. 49. The output of the model supports the dose ranges for the phase2b HS study.

### PK/PD Rationale

An exposure-response (E-R) trend was observed for HiSCR at Weeks 12 and 16 in the Phase 2 study described in Example 4 (FIG. 47), indicating higher doses may achieve higher HiSCR response.

The 350 mg dose may assess the minimum clinical effective dose, the 700 mg dose may generate efficacy and safety data to decrease regulatory risk and increase modeling robustness, and the 1050 mg dose may provide 3-fold higher exposure compared to 350 mg to achieve higher efficacy.

Estimated T_{½} of bermekimab is ~1 week, which supports weekly dosing to maintain adequate drug exposure over the entire dosing interval and does not support less frequent dosing interval.

### Practicality and Subject Burden

Consideration is given to weekly injection burden (<4 injections).

### Supporting Safety for Dose Selection and Safety Management Measures

Bermekimab was well tolerated and safe up to 800 mg SC at Week 0 and 1, followed by 400 mg SC qw and 7.5 mg/kg IV q2w in HS patients (as described in Examples 1 and 4 above). 7.5 mg/kg IV dosing was equivalent to 1125 mg SC dose for a 90 kg individual (which is the average bodyweight in HS patients) assuming SC bioavailability of 60%.

Based on PK simulation, Cₘₐₓ for 1050 mg qw would be lower (0.83-fold) than 7.5 mg/kg IV q2w; and AUC_{2weeks} would be higher (1.85-fold) than 7.5 mg/kg IV q2w.

Safety margins at 700 and 1050 mg SC qw are predicted to be >33 fold based on PK exposure at NOAEL (300 mg/kg SC) in a 1-month toxicology study in cynomolgus monkeys, as shown below:

| Regimen | Cₘₐₓ,ₛₛ (µg/mL) | | AUC_{1week},ₛₛ (µg.day/mL) | |
|---|---|---|---|---|
| | Mean (SD) | Safety Margin | Mean (SD) | Safety Margin |
| 700 mg SC qw | 74.5 (37.8) | 60 | 461.3 (263.6) | 49 |
| 1050 mg SC qw | 111.7 (56.7) | 40 | 691.9 (395.4) | 33 |

Safety margin was calculated based on steady-state (Week 5) PK parameters observed in female cynomolgus monkeys at NOAEL (no-observed-adverse-effect level; 300 mg/kg SC, 6-week non-GLP toxicology study): C_{max,ss} = 4483 µg/mL; AUC_{1week,ss} = 22624 µg.day/mL. PK simulation is based on a preliminary HS population PK model

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating hidradenitis suppurativa in a human subject having lesions associated with hidradenitis suppurativa, the method comprising the step of administering to the subject a pharmaceutical composition comprising a pharmaceutically acceptable carrier and an amount of an anti-IL-1α antibody effective to treat a symptom of hidradenitis suppurativa in the subject, optionally wherein the method comprises administering one or more maintenance doses of the pharmaceutical composition and optionally administering one or more loading doses before administering the first maintenance dose.

2. The method of claim 1, wherein the loading dose is greater than the maintenance dose, for example wherein the loading dose is double the maintenance dose.

3. The method of claim 1 or claim 2, wherein the maintenance dose is about 200 mg or about 350 mg, or about 400 mg or about 700 mg or about 800 mg or about 1050 mg or about 1,200 mg of the anti-IL-1α antibody.

4. The method of any of claims 1-3, wherein the loading dose is about 200 mg or about 350 mg or about 400 mg or about 700 mg or about 800 mg or about 1050 mg or about 1,200 mg of the anti-IL-1α antibody.

5. The method of any of claims 1-4, wherein the one or more maintenance doses are administered once a week.

6. The method of any of claims 1-4, wherein the one or more maintenance doses are administered once every two weeks.

7. The method of any of claims 1-6, wherein the final loading dose is administered one week before the first maintenance dose is administered.

8. The method of any preceding claim, wherein the pharmaceutical composition is administered for at least 12 weeks, at least 13 weeks, at least 14 weeks, at least 15 weeks or at least 16 weeks.

9. The method of claim 8, wherein the pharmaceutical composition is administered for at least 16 weeks.

10. The method of claims 1-7, wherein the pharmaceutical composition is administered for at least 32 weeks.

11. The method of any preceding claim, wherein the administration is subcutaneous or intravenous.

12. The method of any preceding claim, wherein the anti-IL-1α antibody is a monoclonal antibody.

13. The method of claim 12, wherein the monoclonal antibody is an IgG1.

14. The method of claim 13, wherein the monoclonal antibody is MABp1.

15. The method of any preceding claim, wherein the anti-IL-1α antibody comprises
(a) the HCDR1, HCDR2 and HCDR3 of SEQ ID NOs: 5, 6 and 7 respectively, and the LCDR1, LCDR2 and LCDR3 of SEQ ID NOs: 8, 9 and 10 respectively;
(b) the HCDR1, HCDR2 and HCDR3 of SEQ ID NOs: 11, 12 and 13 respectively, and the LCDR1, LCDR2 and LCDR3 of SEQ ID NOs: 14, 15 and 16 respectively; or
(c) the HCDR1, HCDR2 and HCDR3 of SEQ ID NOs: 17, 18 and 19 respectively and the LCDR1, LCDR2 and LCDR3 of SEQ ID NOs: 20, 21 and 22 respectively.

16. The method of any preceding claim, wherein the anti-IL-1α antibody comprises the VH of SEQ ID NO: 3 and the VL of SEQ ID NO: 4.

17. The method of any preceding claim, wherein the subject’s HiSCR score is improved after administration of the pharmaceutical composition.

18. The method of any preceding claim, wherein the median size of the subject’s hidradenitis suppurativa lesions is reduced after administration of the pharmaceutical composition.

19. The method of any preceding claim, wherein the subject’s pain associated with the subject’s hidradenitis suppurativa lesions is reduced after administration of the pharmaceutical composition.

20. The method of any preceding claim, wherein the subject’s time to new hidradenitis suppurativa lesions is increased after administration of the pharmaceutical composition.

21. The method of any preceding claim, wherein the hidradenitis suppurativa in the human subject has failed to resolve after treatment with tumor necrosis factor alpha inhibitors.

22. The method of any preceding claim, further comprising the step of administering to the subject a pharmaceutical composition comprising a pharmaceutically acceptable carrier and an anti-S. aureus antibody.

23. The method of claim 22, wherein the anti-S. aureus antibody comprises a Fab region paratope that specifically binds to S. aureus protein A (SpA) and an Fc region that does not specifically bind SpA.

24. The method of any preceding claim, wherein the concentration of the anti-IL-1α antibody in the pharmaceutical composition is about 100 mg/ml, about 125 mg/ml, about 150 mg/ml, about 175 mg/ml, or about 200 mg/ml.

25. The method of any preceding claim, wherein the subject has moderate to severe hidradenitis suppurativa.

26. The method of any preceding claim, wherein the subject has hidradenitis suppurativa for at least 1 year prior to treatment.
